SEQUENCE LISTING (92 sequences)

SEQ ID NO: 1 - protein, 7 aa, synthetic construct
  MOD_RES 2: note = L-2-naphthylalanine
FXRRRRQ

SEQ ID NO: 2 - protein, 7 aa, synthetic construct
  MOD_RES 2: note = L-2-naphthylalanine
RRXFRRQ

SEQ ID NO: 3 - protein, 7 aa, synthetic construct
  MOD_RES 5: note = L-2-naphthylalanine
RRFRXRQ

SEQ ID NO: 4 - protein, 7 aa, synthetic construct
  MOD_RES 6: note = L-2-naphthylalanine
FRRRRXQ

SEQ ID NO: 5 - protein, 8 aa, synthetic construct
  MOD_RES 2: note = L-2-naphthylalanine
FXRRRRRQ

SEQ ID NO: 6 - protein, 6 aa, synthetic construct
  MOD_RES 2: note = L-2-naphthylalanine
RXRRRQ

SEQ ID NO: 7 - protein, 8 aa, synthetic construct
  MOD_RES 6: note = L-2-naphthylalanine
RFRFRXRQ

SEQ ID NO: 8 - protein, 8 aa, synthetic construct
  source 1..8: note = L-2-naphthylalanine
FFXRRRRQ

SEQ ID NO: 9 - protein, 7 aa, synthetic construct
  MOD_RES 5: note = L-2-naphthylalanine
  MOD_RES 1: note = D-arginine
XRFRXRQ

SEQ ID NO: 10 - protein, 8 aa, synthetic construct
  MOD_RES 1: note = D-phenylalanine
  MOD_RES 4: note = D-arginine
  MOD_RES 6: note = D-arginine
  MOD_RES 2: note = L-2-naphthylalanine
XXRXRXRQ

SEQ ID NO: 11 - protein, 8 aa, synthetic construct
  MOD_RES 3: note = L-2-naphthylalanine
  MOD_RES 5: note = D-arginine
  MOD_RES 7: note = D-arginine
  MOD_RES 2: note = D-phenylalanine
FXXRXRXQ

SEQ ID NO: 12 - protein, 7 aa, synthetic construct
  MOD_RES 3: note = D-arginine
  MOD_RES 5: note = D-arginine
  MOD_RES 2: note = D-2-naphthylalanine
FXXRXRQ

SEQ ID NO: 13 - protein, 7 aa, synthetic construct
FWRRRRQ

SEQ ID NO: 14 - protein, 7 aa, synthetic construct
  MOD_RES 2: note = L-2-naphthylalanine
YXRRRRQ

SEQ ID NO: 15 - protein, 7 aa, synthetic construct
  MOD_RES 2: note = L-2-naphthylalanine
HXRRRRQ

SEQ ID NO: 16 - protein, 7 aa, synthetic construct
  MOD_RES 2: note = L-2-naphthylalanine
  MOD_RES 1: note = L-phenylglycine
XXRRRRQ

SEQ ID NO: 17 - protein, 5 aa, synthetic construct
CMGRC

SEQ ID NO: 18 - protein, 5 aa, synthetic construct
PHSRN

SEQ ID NO: 19 - protein, 4 aa, synthetic construct
LHRD

SEQ ID NO: 20 - protein, 17 aa, synthetic construct
RPRAPARSASRPRRPVE

SEQ ID NO: 21 - protein, 16 aa, synthetic construct
GLRKRLRKFRNKIKEK

SEQ ID NO: 22 - protein, 12 aa, synthetic construct
PLILLRLLRGQF

SEQ ID NO: 23 - protein, 4 aa, synthetic construct
CAQK

SEQ ID NO: 24 - protein, 31 aa, synthetic construct
KDEPQRRSARLSAKPAPPKPEPKPKKAPAKK

SEQ ID NO: 25 - protein, 9 aa, synthetic construct
CGNKRTRGC

SEQ ID NO: 26 - protein, 5 aa, synthetic construct
CREKA

SEQ ID NO: 27 - protein, 9 aa, synthetic construct
CSNRDARRC

SEQ ID NO: 28 - protein, 4 aa, synthetic construct
AHNP

SEQ ID NO: 29 - protein, 12 aa, synthetic construct
YCDGFYACYMDV

SEQ ID NO: 30 - protein, 12 aa, synthetic construct
KQFSALPFNFYT

SEQ ID NO: 31 - protein, 14 aa, synthetic construct
PLGLAGGWGERDGS

SEQ ID NO: 32 - protein, 13 aa, synthetic construct
GGGGYDRVTIHPF

SEQ ID NO: 33 - protein, 7 aa, synthetic construct
VHSPNKK

SEQ ID NO: 34 - protein, 7 aa, synthetic construct
VHPKQHR

SEQ ID NO: 35 - protein, 19 aa, synthetic construct
VLTTGLPALISWIKRKRQQ

SEQ ID NO: 36 - protein, 7 aa, synthetic construct
NNSKSHT

SEQ ID NO: 37 - protein, 12 aa, synthetic construct
VHPKQHRAEEAK

SEQ ID NO: 38 - protein, 10 aa, synthetic construct
CNNSKSHTCC

SEQ ID NO: 39 - protein, 4 aa, synthetic construct
VHPK

SEQ ID NO: 40 - protein, 13 aa, synthetic construct
VHPKQHRGGSKGC

SEQ ID NO: 41 - protein, 9 aa, synthetic construct
CKGGRAKDC

SEQ ID NO: 42 - protein, 13 aa, synthetic construct
CGFECVRQCPERC

SEQ ID NO: 43 - protein, 4 aa, synthetic construct
KDEL

SEQ ID NO: 44 - protein, 6 aa, synthetic construct
SEKDEL

SEQ ID NO: 45 - protein, 17 aa, synthetic construct
MRYMILGLLALAAVCSA

SEQ ID NO: 46 - protein, 12 aa, synthetic construct
RLDPTSYLRTFW

SEQ ID NO: 47 - protein, 6 aa, synthetic construct
WYRGRL

SEQ ID NO: 48 - protein, 26 aa, synthetic construct
ACDCRGDCFCGGKLAKLAKKLAKLAK

SEQ ID NO: 49 - protein, 30 aa, synthetic construct
MSVLTPLLLRGLTGSARRLPVPRAKIHWLC

SEQ ID NO: 50 - protein, 4 aa, synthetic construct
GKRK

SEQ ID NO: 51 - protein, 14 aa, synthetic construct
KLAKLAKKLAKLAK

SEQ ID NO: 52 - protein, 6 aa, synthetic construct
KKKRKV

SEQ ID NO: 53 - protein, 17 aa, synthetic construct
KRPAATKKAGQAKKKKL

SEQ ID NO: 54 - protein, 12 aa, synthetic construct
GRKKRRQRRRPQ

SEQ ID NO: 55 - protein, 8 aa, synthetic construct
RRRRRRRR

SEQ ID NO: 56 - protein, 16 aa, synthetic construct
RQIKIWFQNRRMKWKK

SEQ ID NO: 57 - protein, 21 aa, synthetic construct
GDIMGEWGNEIFGAIAAGFLG

SEQ ID NO: 58 - protein, 4 aa, synthetic construct
GALA

SEQ ID NO: 59 - protein, 30 aa, synthetic construct
WEAALAEALAEALAEHLAEALAEALEALAA

SEQ ID NO: 60 - protein, 7 aa, synthetic construct
FFLIPKG

SEQ ID NO: 61 - protein, 13 aa, synthetic construct
THRPPMWSPWVWP

SEQ ID NO: 62 - protein, 19 aa, synthetic construct
TFFYGGSRGKRNNFKTEEY

SEQ ID NO: 63 - protein, 16 aa, synthetic construct
FKESWREARGTRIERG

SEQ ID NO: 64 - protein, 36 aa, synthetic construct
MCMPCFTTDHQMARKCDDCCGGKGRGKCYGPQCLCR

SEQ ID NO: 65 - protein, 9 aa, synthetic construct
  MOD_RES 9: note = diaminopimelic acid
DLATEPALX

SEQ ID NO: 66 - protein, 7 aa, synthetic construct
GFTGFLS

SEQ ID NO: 67 - protein, 29 aa, synthetic construct
YTIWMPENPRPGTPCDIFTNSRGKRASNG

SEQ ID NO: 68 - protein, 11 aa, synthetic construct
CRGDKRGPDEC

SEQ ID NO: 69 - protein, 36 aa, synthetic construct
TAMRAVDKLLLHLKKLFREGQFNRNFESIIICRDRT

SEQ ID NO: 70 - protein, 9 aa, synthetic construct
CGEMGWVRC

SEQ ID NO: 71 - protein, 12 aa, synthetic construct
  MOD_RES 1: note = 3,4-Dihydroxyphenylacetic acid
XMYIEALDKYAC

SEQ ID NO: 72 - protein, 7 aa, synthetic construct
PKKKRKV

SEQ ID NO: 73 - protein, 10 aa, synthetic construct
AAFEDLRVLS

SEQ ID NO: 74 - protein, 16 aa, synthetic construct
  MOD_RES 11: note = Qln
KRPAATKKRGXRKKKK

SEQ ID NO: 75 - protein, 8 aa, synthetic construct
GPLGIAGQ

SEQ ID NO: 76 - protein, 12 aa, synthetic construct
THRPPMWSPVWP

SEQ ID NO: 77 - protein, 12 aa, synthetic construct
SFHQFARATLAS

SEQ ID NO: 78 - protein, 12 aa, synthetic construct
YEQDPWGVKWWY

SEQ ID NO: 79 - protein, 9 aa, synthetic construct
CSPGAKVRC

SEQ ID NO: 80 - protein, 12 aa, synthetic construct
MEWSLEKGYTIK

SEQ ID NO: 81 - protein, 5 aa, synthetic construct
CALNN

SEQ ID NO: 82 - protein, 5 aa, synthetic construct
CCVVT

SEQ ID NO: 83 - protein, 6 aa, synthetic construct
CLPFFD

SEQ ID NO: 84 - protein, 7 aa, synthetic construct
  MOD_RES 1: note = gamma-Glu
  MOD_RES 3: note = gamma-Glu
  MOD_RES 5: note = gamma-Glu
XCXCXCG

SEQ ID NO: 85 - protein, 15 aa, synthetic construct
GCGGCGGKGGCGGCG

SEQ ID NO: 86 - protein, 21 aa, synthetic construct
GNYTCEVTELTREGETIIELK

SEQ ID NO: 87 - protein, 153 aa, synthetic construct
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 88 - protein, 177 aa, synthetic construct
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 89 - RNA, 534 nt, synthetic construct
  source 1..534: mol_type = other RNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 90 - RNA, 465 nt, synthetic construct
  source 1..465: mol_type = other RNA
atgcggcctagcggcagaaagtctagcaagatgcaggcctttagaatctgggacgttaatcagaaaaccttctacctcagaaacaaccagctggtcgccggctacctgcagggccccaacgtgaatctggaagagaagatcgacgtggtgccaatcgagcctcacgccctgtttctgggcatccacggcggaaaaatgtgcctgtcttgtgtgaagtccggagatgagacaagactgcaactggaagccgtgaacattacagacctgagcgagaaccggaagcaggacaagcggttcgccttcatcagaagcgacagcggccctacaaccagcttcgagagcgccgcttgccccggctggttcctgtgcaccgccatggaagctgatcagcctgtgtccctgaccaacatgcctgatgaaggcgtgatggtgaccaagttctacttccaggaggacgagatgtga

SEQ ID NO: 91 - protein, 8 aa, synthetic construct
GPLGOAGQ

SEQ ID NO: 92 - protein, 10 aa, synthetic construct
GDEVEAPKGC

(19) United States
(12) Patent Application Publication
(10) Pub. No.: US 2025/0367300 A1
(43) Pub. Date: Dec. 4, 2025

(54) Nanomaterial and Methods of Use Thereof

(71) Applicant: University of Connecticut, Farmington, CT (US)

(72) Inventors: Yupeng Chen, Farmington, CT (US); Wuxia Zhang, Farmington, CT (US); Jinhyung Lee, Willimantic, CT (US)

(73) Assignee: University of Connecticut, Farmington, CT (US)

(21) Appl. No.: 18/835,731

(22) PCT Filed: Feb. 3, 2023
(86) PCT No.: PCT/US2023/061943

§ 371 Date: Aug. 4, 2024

Related U.S. Application Data
(60) Provisional application No. 63/306,137, filed on Feb. 3, 2022.

(51) Int. Cl. A61K 47/54; A61K 47/69

(52) U.S. Cl. CPC A61K 47/545; A61K 47/6949

(57) ABSTRACT

A self-assembled nanomaterial includes a Janus base nanotube and a biologically active molecule covalently or non-covalently adhered thereto, wherein the Janus base nanotube includes at least one compound represented by disclosed Formulas I, II, III, IV. V. VI, VII, VIII, or IX, or a pharmaceutically acceptable salt thereof.

### CROSS-REFERENCE TO RELATED APPLICATIONS

The present disclosure claims priority to and the benefit of PCT Application No. PCT/US23/61943, filed Feb. 3, 2023, and U.S. Provisional Patent Application No. 63/306,137, filed 3 Feb. 2022, which is incorporated by reference herein in its entirety.

### FEDERAL RESEARCH STATEMENT

This invention was made with government support under W81XWH-21-1-0274 awarded by the Medical Research and Development Command, AR072027 awarded by the National Institutes of Health, and 1905785, and 2025362 awarded by the National Science Foundation. The government has certain rights in the invention.

### INCORPORATION BY REFERENCE

In compliance with 37 C.F.R. 1.52 (e) (5), the sequence information contained in electronic file name: EASC003PCTUS.SeqList1_ST26.xml; size 91.2 KB; created Jan. 23, 2025, is incorporated herein by reference in its entirety.

### BACKGROUND

Numerous methods of delivering biologically active materials have been developed and vary based upon the type and the target of the biologically active material. Examples of various delivery vehicles which have been used include virus vectors, lipid nanoparticles, cationic polymers, liposomes, polymer nanoparticles, carbon nanoparticles, superparamagnetic iron oxide nanoparticles (SPION), gold nanoparticles, silver nanoparticles, metal-organic frameworks, cell-penetrating peptides, black phosphorus nanosheets, and DNA nanostructures. Delivery vectors such as lipid nanoparticles can deliver RNAs but report a low endosomal escape, which reduces efficacy. Moreover, cationic lipid content tends to drive a pro-inflammatory phenotype. These limitations make it challenging to obtain high efficacy for therapeutic applications, and have impeded translation into clinics.

There remains a need for improved delivery of biologically active materials such as small molecules, nucleic acids, proteins and gene silencing or gene editing tools, and/or other large molecule biological materials.

### SUMMARY

Disclosed herein is a self-assembled nanomaterial comprising a Janus base nanotube and a biologically active molecule covalently or non-covalently adhered thereto, wherein the Janus base nanotube comprises at least one compound represented by Formulas I, II, III, IV, V, VI, VII, VIII, or IX, or a pharmaceutically acceptable salt thereof:

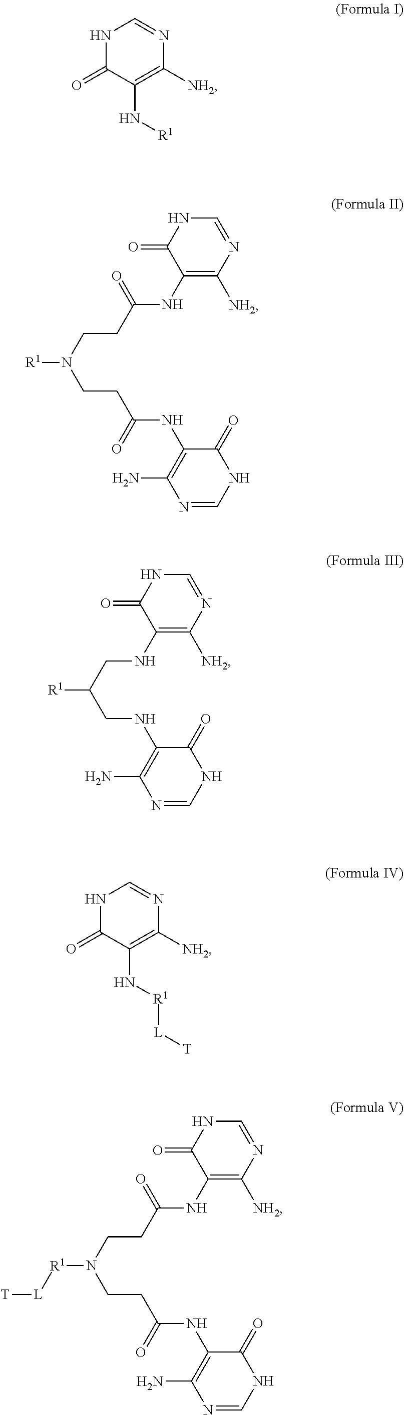

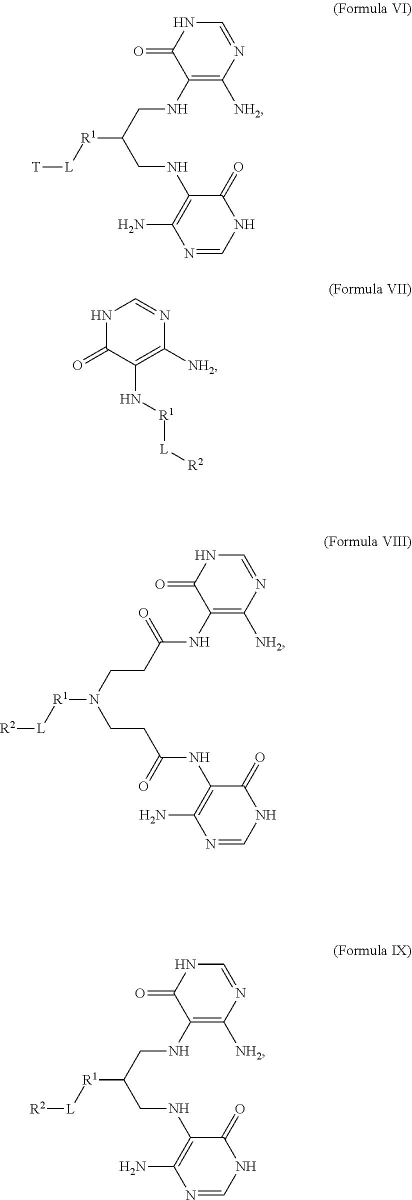

wherein
R¹ for Formulas I-III is H, —(CH₂)ⱼH, —(CH₂CH₂O)ₖH, —(CH₂CH₂NH)ₘH, an α-amino acid, a β-amino acid, an α-polypeptide, or a β-polypeptide;
R¹ of Formulas IV-IX is absent, —(CH₂)ⱼ—, —(CH₂CH₂O)ₖ—, —(CH₂CH₂NH)ₘ—, an α-amino acid, a β-amino acid, an α-polypeptide, or a β-polypeptide;
each j, k and m is independently 1-200;
L is a bond or a linker group;
T is H, a biologically active molecule (e.g., an agent, such as a therapeutic agent), or a targeting molecule; and
R² is H or a coating/protecting material.

Also disclosed herein are injectable compositions comprising the self-assembled nanomaterials described above and a pharmaceutically acceptable carrier.

The above described and other features are exemplified by the following figures and detailed description.

### BRIEF DESCRIPTION OF THE DRAWINGS

The following figures are exemplary embodiments wherein the like elements are numbered alike.

FIGS. 1A, 1B, and 1C are transmission electron microscope (TEM) images of self-assembled, single ring nanotubes. FIG. 1A shows the self-assembled nanomaterial composed of compound S4 having a single ring nanotube structure. FIG. 1B shows the self-assembled nanomaterial composed of compound S10, a structure including twin single rings. FIG. 1C shows the self-assembled nanomaterial composed of compound S17. Scale bar: 100 nm.

FIGS. 2A, 2B, 2C, and 2D are TEM images of Janus Base Nanopiece encapsulating mRNA (JBNP-mRNA) and demonstrate cellular delivery. FIG. 2A shows the transmission electron microscope (TEM) image of JBNP-mRNA. FIG. 2B shows the hydrodynamic size of the JBNP-mRNAs. FIG. 2C shows the surface charge of JBNP-mRNAs.

FIG. 2D shows the delivery of mRNAs to the human chondrocytes (C28/12).

### DETAILED DESCRIPTION

Disclosed herein are self-assembled nanomaterials comprising Janus nanotubes which are composed of units having a single ring system. The self-assembled nanomaterials have low cytotoxicity, low immunogenicity, and demonstrate minimal side effects in vivo, and may be advantageously used to deliver a biologically active material. The self-assembled nanomaterials also demonstrate improved endosomal escape leading to high efficacy.

In any aspect or embodiment described herein, the self-assembled nanomaterials disclosed herein can be a delivery vehicle for small molecules, genes, and protein delivery to cells.

The self-assembled nanomaterials disclosed herein may include a targeting molecule or moiety targeting specific receptors on the surface of various cells to facilitate specific and selective uptake. The co-assembly of different Janus nanotubes having different functional groups, and their use in differing amounts, allows for the design of self-assembled nanomaterials having desired properties. These properties may be adjusted based upon cellular delivery, circulation time in vivo, passive or active targeting, subcellular targeting, improved cellular uptake, and enhanced endosomal escape.

Throughout the present specification and the accompanying claims the words “comprise,” “include,” and “have” and variations thereof, such as “comprises,” “comprising,” “includes,” “including,” “has,” and “having”, are to be interpreted inclusively. That is, these words are intended to convey the possible inclusion of other elements or integers not specifically recited, where the context allows. No language in the specification should be construed as indicating any non-claimed element essential to the practice of the invention.

The terms “a,” “an,” and “the” and similar referents used in the context of describing the invention (especially in the context of the following claims) are to be construed to cover both the singular and the plural, unless otherwise indicated herein or clearly contradicted by context.

The terms first, second, etc. as used herein are not meant to denote any particular ordering, but simply for convenience to denote a plurality of, for example, layers.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their System International de Unites (SI) accepted form.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the invention and are not intended to limit the invention thereto.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +10% or 5% of the stated value.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

As used herein, the term “administering” means the actual physical introduction of a composition into or onto (as appropriate) a host or cell. Any and all methods of introducing the composition into the host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopcia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

### Chemical Definitions

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

A pharmaceutically acceptable salt, in any aspect or embodiment described herein, includes salts that retain the biological effectiveness and properties of the compound, and which are not biologically or otherwise undesirable. Pharmaceutically acceptable base addition salts can be prepared from inorganic and organic bases. In any aspect or embodiment described herein, salts derived from inorganic bases include, by way of example only, sodium, potassium, lithium, ammonium, calcium and magnesium salts. In any aspect or embodiment described herein, salts derived from organic bases include, but are not limited to, salts of primary, secondary and tertiary amines, such as alkyl amines, dialkyl amines, trialkyl amines, substituted alkyl amines, di (substituted alkyl)amines, tri (substituted alkyl)amines, alkenyl amines, dialkenyl amines, trialkenyl amines, substituted alkenyl amines, di (substituted alkenyl)amines, tri (substituted alkenyl)amines, cycloalkyl amines, di(cycloalkyl) amines, tri (cycloalkyl)amines, substituted cycloalkyl amines, disubstituted cycloalkyl amine, trisubstituted cycloalkyl amines, cycloalkenyl amines, di(cycloalkenyl) amines, tri (cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

In any aspect or embodiment described herein, salts derived from organic bases include, but are not limited to, salts of primary, secondary and tertiary amines, such as alkyl amines, dialkyl amines, trialkyl amines, substituted alkyl amines, di (substituted alkyl)amines, tri (substituted alkyl)amines, alkenyl amines, dialkenyl amines, trialkenyl amines, substituted alkenyl amines, di (substituted alkenyl)amines, tri (substituted alkenyl)amines, cycloalkyl amines, di(cycloalkyl) amines, tri (cycloalkyl)amines, substituted cycloalkyl amines, disubstituted cycloalkyl amine, trisubstituted cycloalkyl amines, cycloalkenyl amines, di(cycloalkenyl) amines, tri (cycloalkenyl)amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

In any aspect or embodiment described herein, examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, in any aspect or embodiment described herein, conventional non-toxic acid salts include those derived from inorganic acids, such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The term “amino acid” refers to a molecule containing both an amino group and a carboxyl group. Exemplary amino acids include, without limitation, both the D- and L-isomers of the naturally-occurring amino acids, as well as non-naturally occurring amino acids prepared by organic synthesis or other metabolic routes. The term amino acid, as used herein, includes without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | | Side-chain | |
|---|---|---|---|---|---|
| | Letter | Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof. “Charged amino acids” are lysine, arginine, histidine, aspartate, glutamate, and analogs thereof.

The term “amino acid analog” refers to a molecule which is structurally similar to an amino acid and that can be substituted for an amino acid in the formation of a peptidomimetic macrocycle. Amino acid analogs include, without limitation, 3-amino acids, and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid that is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

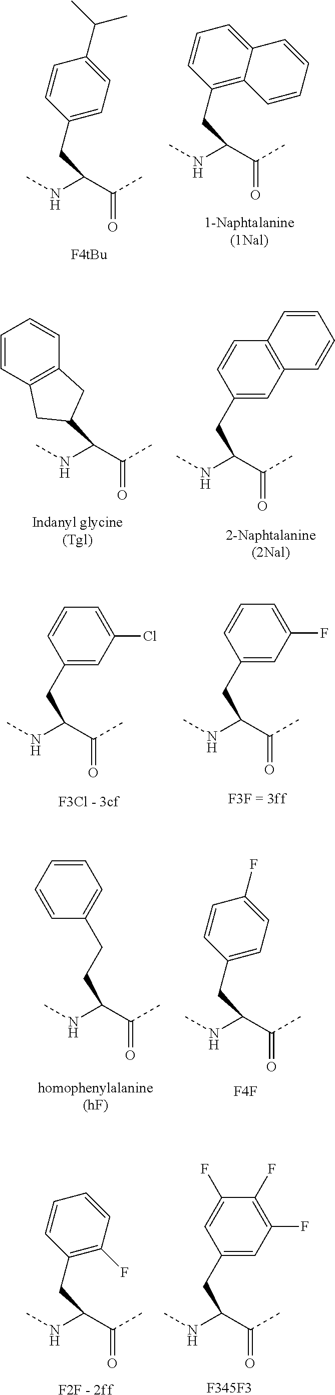

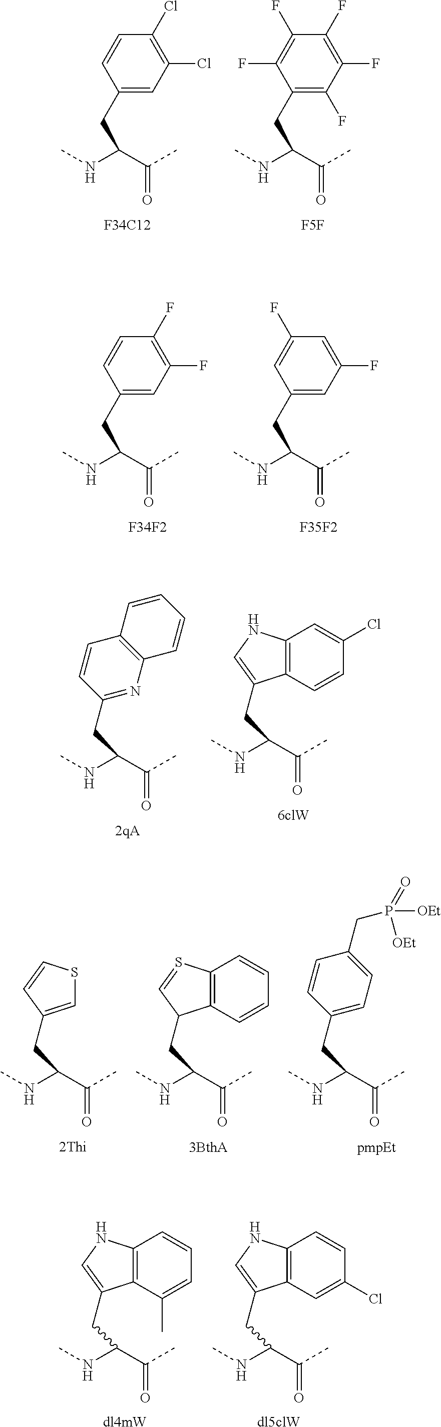

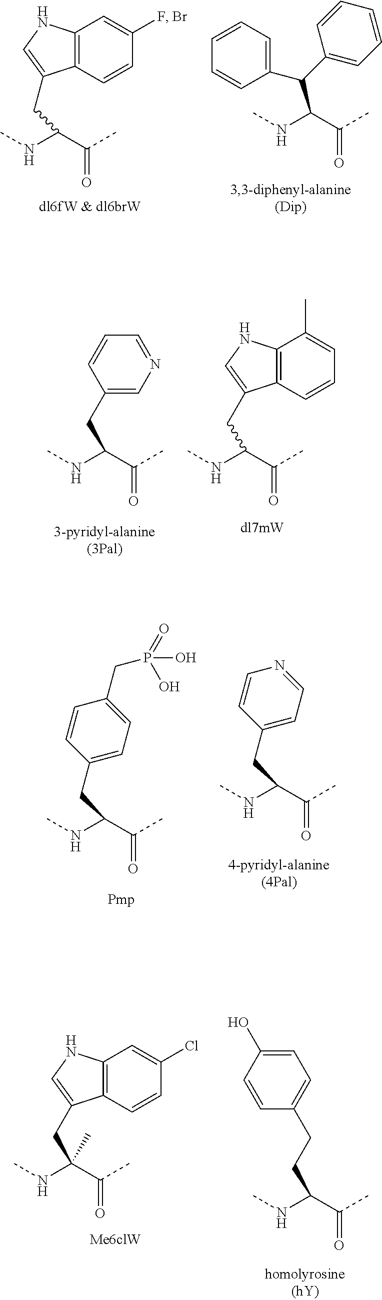

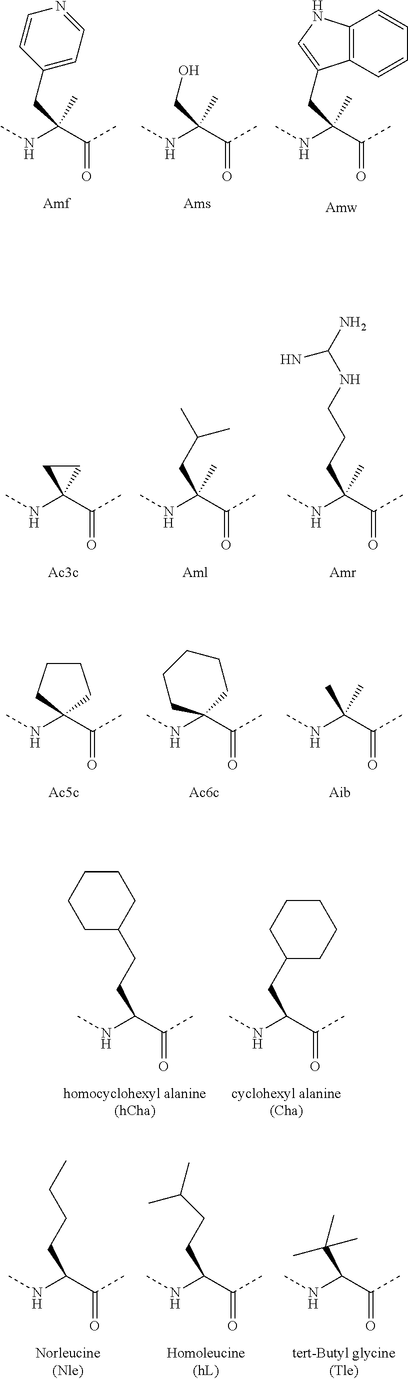

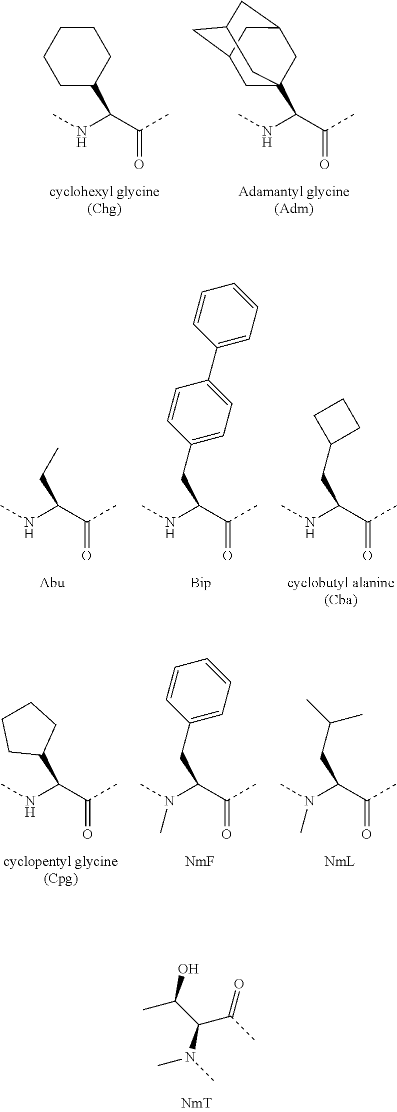

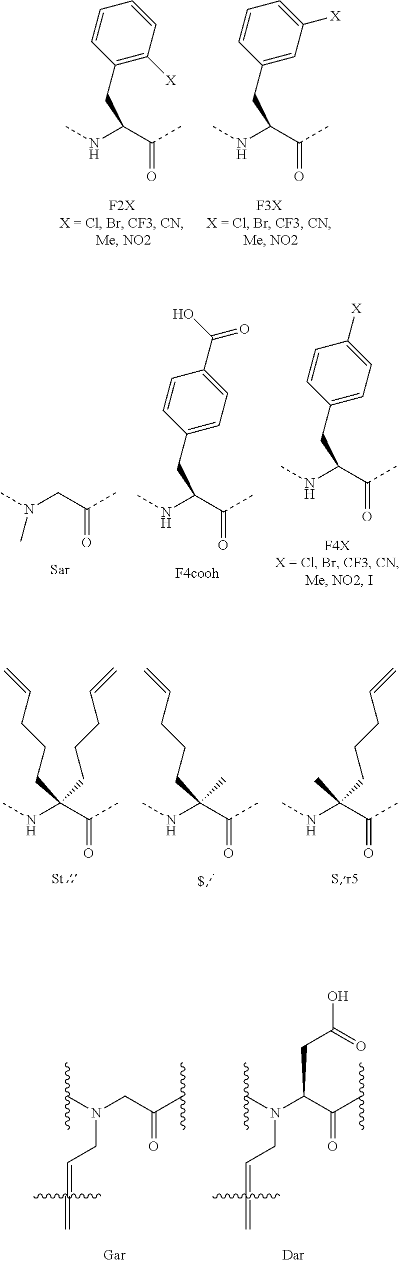

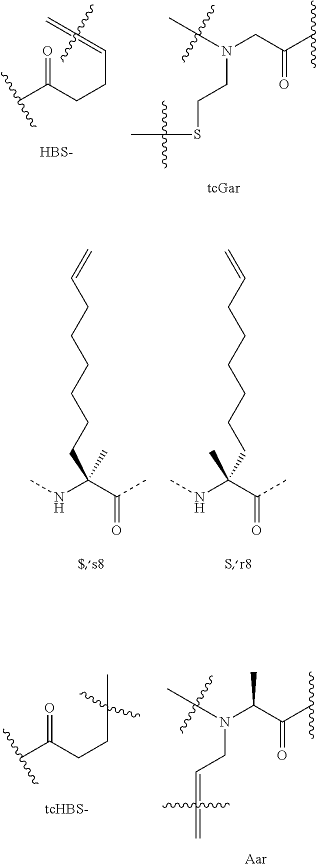

In any aspect or embodiment descried herein, amino acid analogs include β-amino acid analogs. In any aspect or embodiment described herein, examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl) butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl) butyric acid; (R)-3-amino-4-(3,4-difluorophenyl) butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thicnyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromcthylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl) butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl) butyric acid; (S)-3-amino-4-(3,4-difluorophenyl) butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homolcucine; L-β-homomethionine; L-β-homophenylalanine; L-β-homoproline; L-β-homotryptophan; L-β-homovaline; L-Nω-benzyloxycarbonyl-β-homolysine; No-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothrconinc; O-t-butyl-L-β-homotyrosine; 2-aminocyclopentane carboxylic acid; and 2-aminocyclohexane carboxylic acid.

In any aspect or embodiment described herein, amino acid analogs include analogs of alanine, valine, glycine or leucine. In any aspect or embodiment described herein, examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; 1-(2-pyridyl)-D-alanine; β-(2-pyridyl)-L-alanine; β-(2-thicnyl)-D-alanine; β-(2-thienyl)-L-alanine;-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; 1-chloro-L-alanine; 1-cyano-L-alanin; 3-cyclohexyl-D-alanine; 3-cyclohexyl-L-alanine; 3-cyclopenten-1-yl-alanine; 3-cyclopentyl-alanine; 3-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valinc; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvalinc; D-phenylglycine; β-aminobutyric acid; β-aminoisobutyric acid; (2-bromophenyl)glycinc; (2-methoxyphenyl)glycine; (2-methylphenyl)glycine; (2-thiazoyl)glycinc; (2-thicnyl)glycine; 2-amino-β-(dimethylamino)-propionic acid; L-α,β-diaminopropionic acid; L-α-aminobutyric acid; L-α-t-butylglycine; L-β-thienyl)glycine; L-2-amino-β-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α, γ-diaminobutyric acid; L-α, γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidenc)ethyl)-D-α,β-diaminopropionic acid; (N-β-1-(4,4-dimethyl-2,6-dioxocyclohex-1-ylidenc)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidenc)ethyl)-L-α,γ-diaminobutyric acid; (N-γ-4-methyltrityl)-D-α,γ-diaminobutyric acid; (N-γ-4-methyltrityl)-L-α, γ-diaminobutyric acid; (N-γ-allyloxycarbonyl)-L-α, γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalaninc; and N-(2-hydroxy-4-methoxy-Bzl)-Gly-OH.

In any aspect or embodiment described herein, amino acid analogs include analogs of arginine or lysine. In any aspect or embodiment described herein, examples of amino acid analogs of arginine and lysine include, but are not limited to, the following: citrulline; L-2-amino-3-guanidinopropionic acid; L-2-amino-3-urcidopropionic acid; L-citrulline; Lys (Mc) 2-OH; Lys(N₃)—OH; Nδ-benzyloxycarbonyl-L-omithine; No-nitro-D-arginine; No-nitro-L-arginine; α-methyl-omithinc; 2,6-diaminoheptanedioic acid; L-omithine; (Nδ-1-(4,4-dimethyl-2,6-dioxo-cyclohex-1-ylidenc)ethyl)-D-omithine; (Nδ-1-(4,4-dimethyl-2,6-dioxo-cyclohex-1-ylidene)ethyl)-L-omithine; (Nδ-4-methyltrityl)-D-omithine; (Nδ-4-methyltrityl)-L-omithin; D-omithine; L-omithine; Arg (Mc) (Pbf)-OH; Arg (Me) 2-OH (asymmetrical); Arg (Me) 2-OH (symmetrical); Lys (ivDde)-OH; Lys (Me) 2-OH.HCl; Lys (Me3)-OH chloride; No-nitro-D-arginine; and No-nitro-L-arginine.

In any aspect or embodiment described herein, amino acid analogs include analogs of aspartic or glutamic acids. In any aspect or embodiment described herein, examples of amino acid analogs of aspartic and glutamic acids include, but are not limited to, the following: α-methyl-D-aspartic acid; α-methyl-glutamic acid; α-methyl-L-aspartic acid; Y-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; Y-carboxy-D-glutamic acid γ,γ-di-t-butyl ester; γ-carboxy-L-glutamic acid γ,γ-di-t-butyl ester; Glu (OAll)-OH; L-Asu (OtBu)-OH; and pyroglutamic acid.

In any aspect or embodiment described herein, amino acid analogs include analogs of cysteine and methionine. In any aspect or embodiment described herein, examples of amino acid analogs of cysteine and methionine include, but are not limited to, Cys (farnesyl)-OH, Cys (farnesyl)-OMe, α-methyl-methionine, Cys (2-hydroxyethyl)-OH, Cys (3-aminopropyl)-OH, 2-amino-4-(ethylthio) butyric acid, buthionine, buthioninesulfoximine, cthionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminocthyl)-L-cysteine, seleno-L-cystine, cystathioninc, Cys (StBu)-OH, and acetamidomethyl-D-penicillaminc.

In any aspect or embodiment described herein, amino acid analogs include analogs of phenylalanine and tyrosine. In any aspect or embodiment described herein, examples of amino acid analogs of phenylalanine and tyrosine include 3-methyl-phenylalanine, 3-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chlorocthyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyroninc, ethyl-tyrosine, and methyl-tyrosine.

In any aspect or embodiment described herein, amino acid analogs include analogs of proline. In any aspect or embodiment described herein, examples of amino acid analogs of proline include, but are not limited to, 3,4-dehydro-proline, 4-fluoro-proline, cis-4-hydroxy-proline, thiazolidine-2-carboxylic acid, and trans-4-fluoro-prolinc.

In any aspect or embodiment described herein, amino acid analogs include analogs of serine and threonine. In any aspect or embodiment described herein, examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-cthoxypropionic acid, 4-amino-3-hydroxybutanoic acid, and α-methylserine.

In any aspect or embodiment described herein, amino acid analogs include analogs of tryptophan. In any aspect or embodiment described herein, examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In any aspect or embodiment described herein, amino acid analogs are racemic. In any aspect or embodiment described herein, the D isomer of the amino acid analog is used. In any aspect or embodiment described herein, the L isomer of the amino acid analog is used. In any aspect or embodiment described herein, the amino acid analog comprises chiral centers that are in the R or S configuration. In any aspect or embodiment described herein, the amino group(s) of β-amino acid analog is substituted with a protecting group, e.g., tert-butyloxycarbonyl(BOC group), 9-fluorenylmethyloxycarbonyl (FMOC), tosyl, and the like. In any aspect or embodiment described herein, the carboxylic acid functional group of a β-amino acid analog is protected, e.g., as its ester derivative. In any aspect or embodiment described herein, the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g. norleucine for methionine) or other properties (e.g., 2-thienylalanine for phenylalanine).

The term “peptide” refers to one or more amino acid residues which are bonded together. The term “polypeptide” refers to a linear organic polymer consisting of a large number of amino-acid residues (20 or more) bonded together in a chain, forming part of (or the whole of) a protein molecule.

The term “α-polypeptide” refers to are polypeptides derived from α-amino acids.

The term “β-polypeptide” refers to are polypeptides derived from β-amino acids.

The term “cyclic peptide” refers to polypeptide chains which contain a circular sequence of bonds. For example, in any aspect or embodiment described herein, cyclic peptides include, without limitation, structures according to the following:

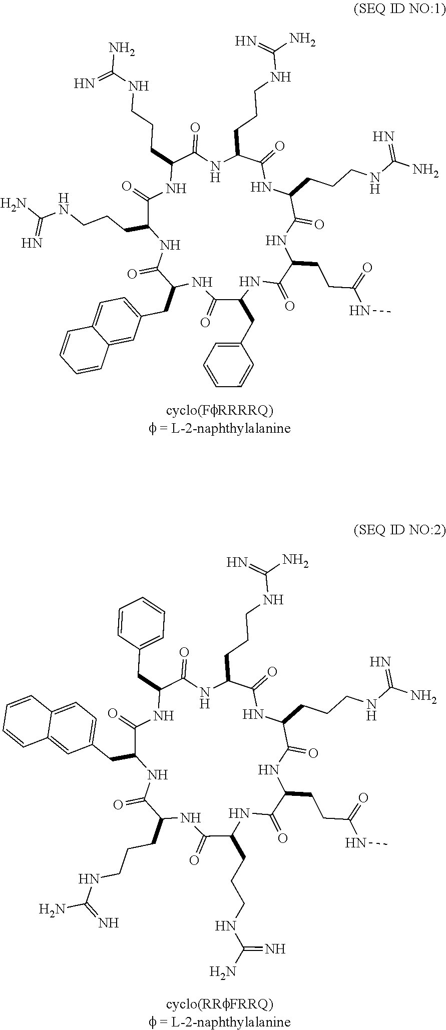

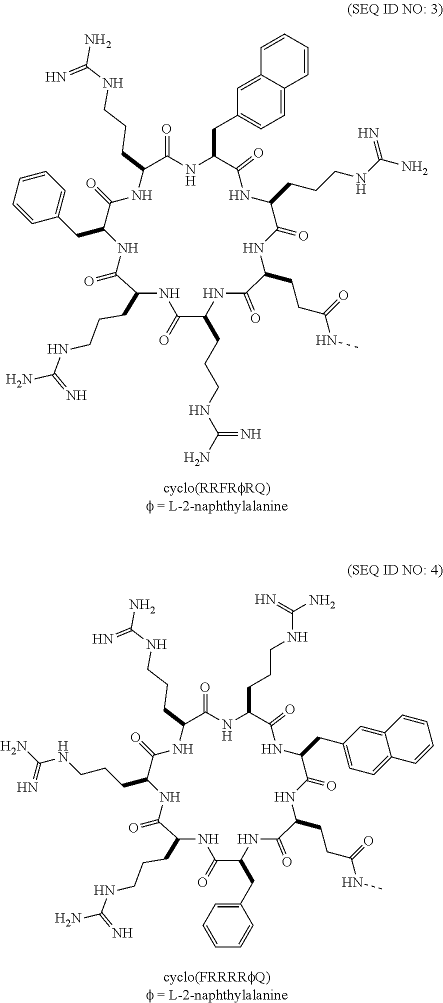

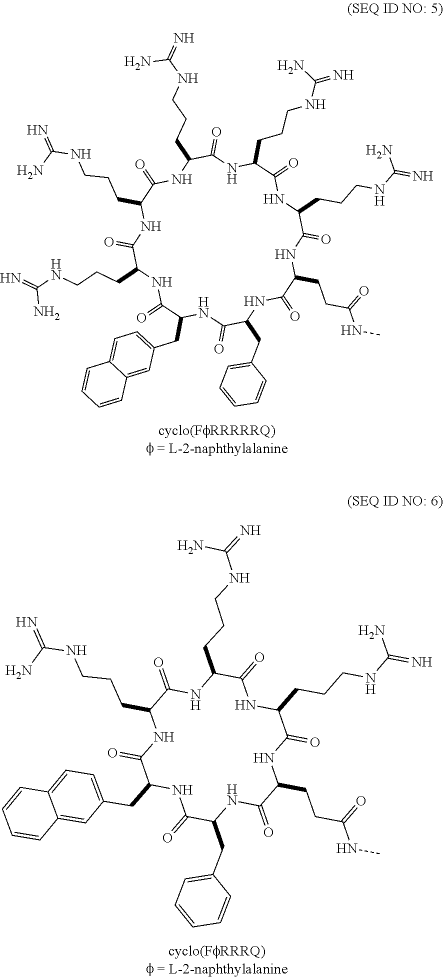

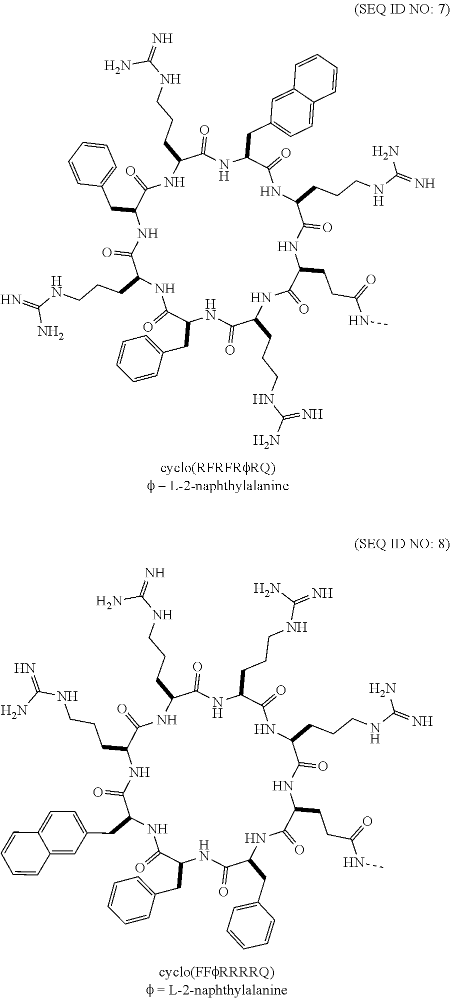

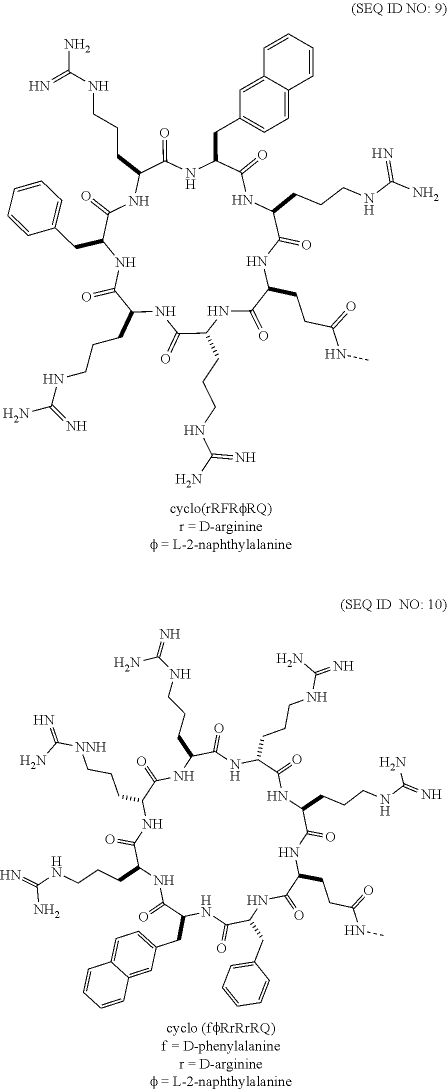

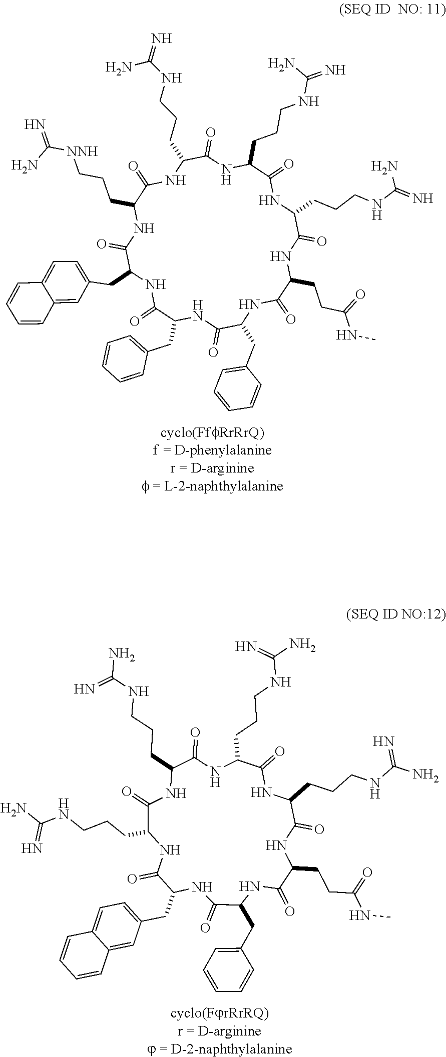

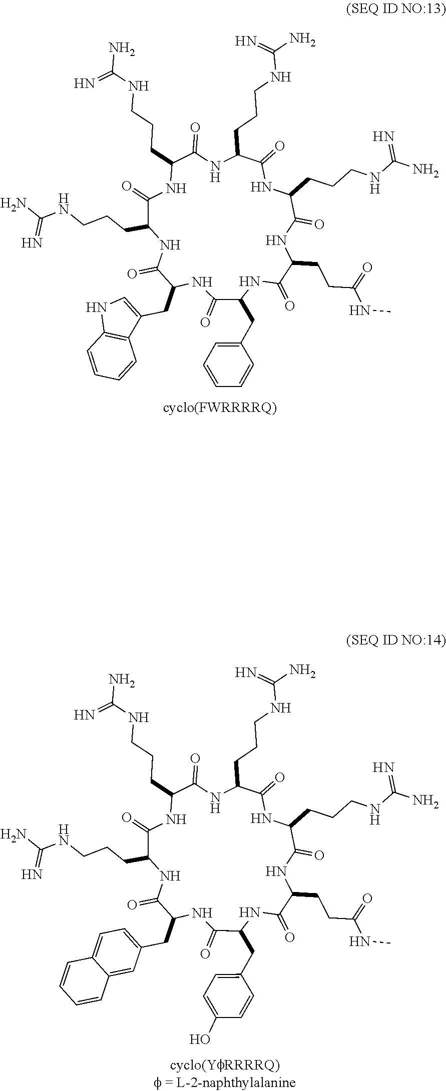

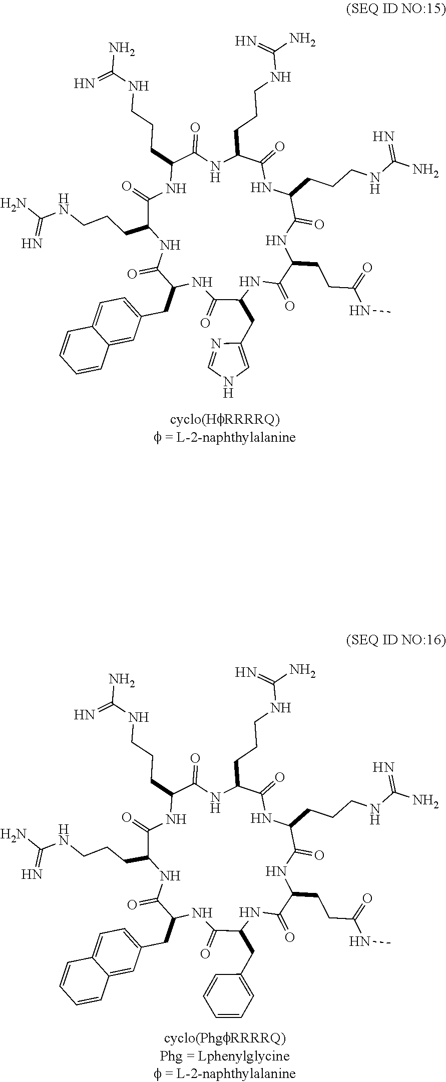

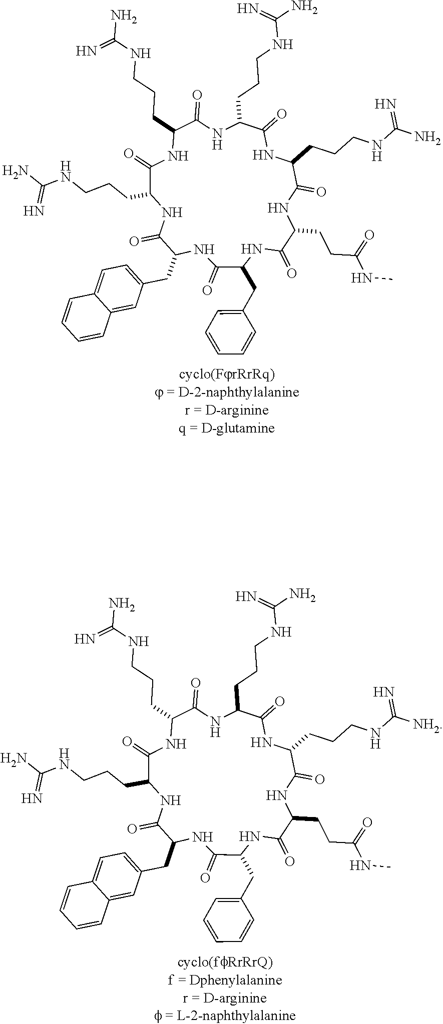

The term “phosphate ester” refers to esters of phosphoric acid, a central phosphate molecule with alkyl or aromatic substituents. For example, in any aspect or embodiment described herein, the phosphate esters include, without limitation, structures according to the following:

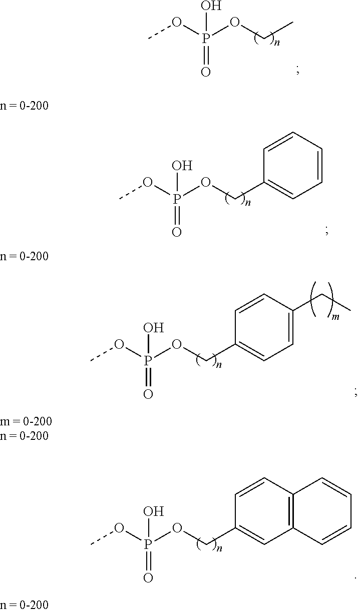

The term “aliphatic” or “aliphatic group” refers to a hydrocarbon moiety that may be straight-chain (i.e., unbranched), branched, or cyclic (including fused, bridging, and spiro-fused polycyclic) and may be completely saturated or may contain one or more units of unsaturation. Suitable aliphatic groups include, but are not limited to, linear or branched, alkyl, alkenyl, and alkynyl groups, and hybrids thereof. As used herein the terms “aliphatic” or “aliphatic group”, also encompass partially substituted analogs of these moieties where at least one of the hydrogen atoms of the aliphatic group is replaced by an atom that is not carbon or hydrogen.

The term “linker” refers to a chemical group that connects one or more other chemical groups via at least one covalent bond.

While the invention has been described with reference to an exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the appended claims. Any combination of the described elements in all possible variations thereof is encompassed by the invention unless otherwise indicated herein or otherwise clearly contradicted by context.

### Self-Assembled Nanomaterial

The self-assembled nanomaterial of the present disclosure comprises a Janus base nanotube (JBNT) and a biologically active molecule (e.g., an agent, such as a therapeutic agent) covalently or non-covalently adhered to the JBNT. In any aspect or embodiment described herein, the JBNT is composed of structural units based on single ring system, and self-assembles into a nanomaterial which can be used for drug delivery and scaffolding. In particular, in any aspect or embodiment described herein, the JBNT provides a solution for the delivery of a biologically active molecule (e.g., an agent, such as a therapeutic agent) to a specific cell and/or tissue. In any aspect or embodiment described herein, the JBNT is coupled/conjugated to a targeting molecule or moiety in order to facilitate active targeting of the self-assembled nanomaterial including the biologically active molecule (e.g., an agent, such as a therapeutic agent) to a specific cell and/or tissue.

The JBNT is a biocompatible, biodegradable material having relatively low cytotoxicity and low immunogenicity. The JBNT also combines the advantages of lipid nanoparticles and cationic polymers for improved endosomal escape and high efficacy. Advantageously, the JBNT efficiently enters cells via macropinocytosis (same mechanism as lipid nanoparticles), and can effectively escape from endosomes via the “proton sponge” effect, which is the same mechanism as cationic polymers. Therefore, the JBNT can achieve excellent delivery of a biologically active molecule (e.g., an agent, such as a therapeutic agent) and present extremely low cytotoxicity.

In any aspect or embodiment described herein, the JBNT comprises at least one compound represented by Formulas I, II, III, IV, V, VI, VII, VIII, IX, or a pharmaceutically acceptable salt thereof.

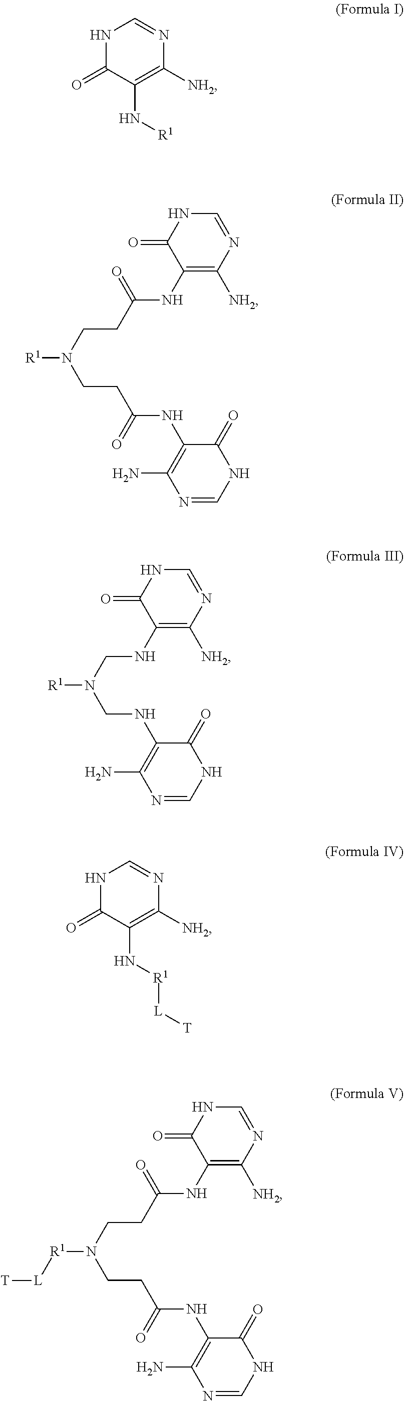

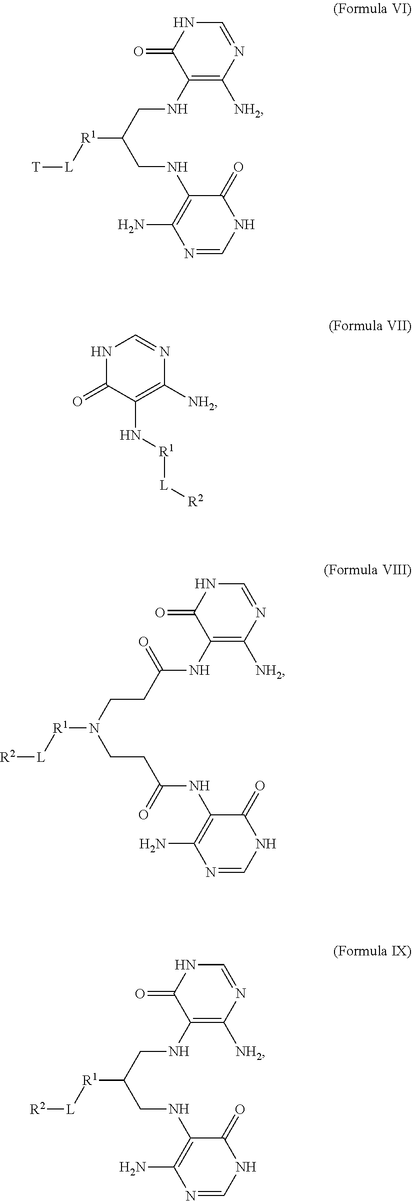

wherein, R¹ for Formulas I-III is H, —(CH₂)ⱼH, —(CH₂CH₂O)ₖH, —(CH₂CH₂NH)ₘH, an α-amino acid, α-amino acid, an α-polypeptide, or a β-polypeptide; R¹ of Formulas IV-IX absent, —(CH₂)ⱼ—, —(CH₂CH₂O)ₖ—, —(CH₂CH₂NH)ₘ—, an α-amino acid, a β-amino acid, an α-polypeptide, or a β-polypeptide; each j, k, and m is independently 1-200; L is a bond or a linker group; T is H, a biologically active molecule (e.g., an agent, such as a therapeutic agent), or a targeting molecule; and R² is H or a coating/protecting material.

In any aspect or embodiment described herein, R¹ of any one of Formula I, II, or III is independently H, —(CH₂)ⱼH, —(CH₂CH₂O)_{K}H, —(CH₂CH₂NH)ₘH, an α-amino acid, a β-amino acid, an α-polypeptide, or a β-polypeptide, wherein each j, k, and m is independently 1-200. In any aspect or embodiment described herein, R¹ of any one of Formula I or III is independently —(CH₂)ⱼH, —(CH₂CH₂O)_{K}H, —(CH₂CH₂NH)ₘH, an α-amino acid, a β-amino acid, an α-polypeptide, or a β-polypeptide, wherein each j, k, and m is independently 1-200. In any aspect or embodiment described herein, R¹ of any one of Formula I, II, or III is independently —(CH₂)ⱼH, —(CH₂CH₂O)ₖH, —(CH₂CH₂NH)ₘ·H, an α-amino acid, a β-amino acid, an α-polypeptide, or a β-polypeptide, wherein each j, k, and m is independently 1-200.

In any aspect or embodiment described herein, R¹ of any one of Formula IV, V, VI, VII, VIII, and IX is independently absent, —(CH₂)ⱼ—, —(CH₂CH₂O)ₖ—, —(CH₂CH₂NH)ₘ—, an α-amino acid, a β-amino acid, an α-polypeptide, or a β-polypeptide.

In any aspect or embodiment described herein, L is the linker group, and is selected from an acid-cleavable group, a reducible disulfide group, an α-amino acid, a β-amino acid, an α-polypeptide, a β-polypeptide, a trans-cyclooctene group, a thioether-containing group, an enzyme cleavable group, a stimuli-responsive group, or a combination thereof. The acid-cleavable linker can be N-acyl hydrazone, a carbonate group, or an ester group. The reducible disulfide linker can be N-succinimidyl-4-(2-pyridyldithio) pentanoate (SPP), N-succinimidyl-4-(2-pyridyldithio) butyrate (SPDB), or 4-(4′-acctylphenoxy) butanoic acid (AcBut), Val-Cit dipeptide, Phc-Lys dipeptide, an α-methyl substituted disulfide, an engineered cysteine residue, or a thiol-containing maytansinoid. The stimuli-responsive linker can be a trans-cyclooctene linker, or a thioether-containing linker. The enzyme cleavable linker can be GPLGOAGQ (SEQ ID NO: 91), GDEVEAPKGC (SEQ ID NO:92), a glycosidase-cleavable linker, a β-glucoronidase-cleavable linker, a β-Galactosidase-cleavable linker, a phosphatase-cleavable linker, a pyrophosphate-containing linker, a dipeptide-containing linker, Phe-Lys-PABC (para-aminobenzyl carbamate), a Val-Cit-PABC containing linker, a citrulline-valine containing linker, a Glu-Val-Cit-containing linker, or a Val-Ala containing linker.

In any aspect or embodiment described herein, T is a targeting molecule and is selected from a small molecule, a polymer (such as an amphiphilic polymer), an aptamer, a peptide, a protein, a polysaccharide, a polyunsaturated fatty acid, a carbohydrate, or a combination thereof. The selection of the targeting molecule depends upon the cell and/or tissue to which the self-assembled nanomaterial is to be delivered.

In any aspect or embodiment described herein, non-limiting examples of the targeting molecule include: (i) small molecules such as folic acid, thiamine, and dimercaptosuccinic acid; (ii) proteins such as bovine serum albumin (BSA), transferrin, antibodies, nanobodies, lectins, cytokines, fibrinogen, and thrombin; (iii) polysaccharides such as hyaluronic acid, chitosan, dextran, oligosaccharides, and heparin; (iv) polyunsaturated fatty acids such as palmitic acid and phospholipids; (v) targeting molecules for infected cells/tissue targeting molecules such as RGD, c (CMGRC) (SEQ ID NO:17), PHSRN (SEQ ID NO:18), LHRD (SEQ ID NO:19), antigenic peptides, internalization peptides, cell-penetrating peptides, VP22, RPRAPARSASRPRRPVE (SEQ ID NO:20), sC18, GLRKRLRKFRNKIKEK (SEQ ID NO: 21), Pept1, and PLILLRLLRGQF (SEQ ID NO:22); (vi) targeting molecules for blood-brain barrier (BBB) penetration such as transferrin, OX26, CAQK (SEQ ID NO:23), and lactoferrin; (vii) tumor targeting molecules such as F3, KDEPQRRSARLSAKPAPPKPEPKPKKAPAKK (SEQ ID NO:24), Lyp-1, CGNKRTRGC (SEQ ID NO:25), CREKA (SEQ ID NO:26), Bld-3, CSNRDARRC (SEQ ID NO:27), AHNP (SEQ ID NO:28), YCDGFYACYMDV (SEQ ID NO:29), SP204 (KQFSALPFNFYT peptide; SEQ ID NO:30), EGF, VEGF, LFA-1, and apolipoprotein AI; (viii) targeting molecules for infarcted cardiac tissue and/or atherosclerotic-related disease such as SP204 (KQFSALPFNFYT; SEQ ID NO:30), PLGLAGGWGERDGS (SEQ ID NO:31), GGGGYDR VTIHPF (SEQ ID NO: 32), VHSPNKK (SEQ ID NO:33), VHPKQHR (SEQ ID NO:34), VLTTGLPALISWIKRKRQQ (SEQ ID NO:35), NNSKSHT (SEQ ID NO:36), VHPKQHRAEEAK (SEQ ID NO:37), C*NNSKSHTC*C (SEQ ID NO:38), VHPK (SEQ ID NO: 39), VHPKQHRGGSKGC (SEQ ID NO:40), Ab (429), antibody specific for VCAM-1 (e.g., Ab M/K-2.7), PECAM-1, ICAM-1 (Ab R6.5), or LFA-1 Integrin; (ix) white fat targeting molecules, such as SP204 and CKGGRAKDC (SEQ ID NO:41); (x) alveoli targeting molecules such as WGA; (xi) intestinal targeting protein such as UEA-1; (xii) membrane dipeptidase targeting molecules, such as GFE and CGFECVRQCPERC (SEQ ID NO:42); (xiii) endoplasmic reticulum (ER) targeting molecules, such as KDEL (SEQ ID NO:43) peptide, SEKDEL (SEQ ID NO: 44), Eriss, and MRYMILGLLALAAVCSA (SEQ ID NO:45) peptide; (xiv) chondrocyte targeting peptides, such as RLDPTSYLRTFW (SEQ ID NO:46); (xv) cartilage targeting peptides, such as WYRGRL (SEQ ID NO:47); (xvi) mitochondrial membrane targeting molecules, such as RGD-4C-GG-D (KLAKLAK) 2 (SEQ ID NO:48), D-Arg-Dmt-Lys-Phc-NH2, Phc-D-Arg-Phe-Lys-NH2, D-Arg-Dmt-Orn-Phe-NH2,D-Arg-(2′6′-dimethylTyr)-Lys-Phe-NH2, (1,7-bis-4-hydroxy-3-methoxyphenyl-1,6-heptadiene-3,5-dione)-triphenyl-phospine, 1,5-dioctadecyl-L-glutamyl 2-histidly-hexahydrobenzoic acid-SPC-L, MSVLTPLLLRGLTGSARRLPVPRAKIHWLC (SEQ ID NO:49), GKRK (SEQ ID NO:50), and D [KLAKLAK]2 (SEQ ID NO:51); (xvii) nucleus targeting molecules, such as KKKRKV (SEQ ID NO:52), KRPAATKKAGQAKKKKL (SEQ ID NO:53), HIV-1 TAT, GRKKRRQRRRPQ (SEQ ID NO:54), R⁸, RRRRRRRR (SEQ ID NO:55), penetratin, RQIKIWFQNRRMKWKK (SEQ ID NO:56), HA2 peptide, GDIMGEWGNEIFGAIAAGFLG (SEQ ID NO:57), GALA (SEQ ID NO:58), WEAALAEALAEALAEHLAEALAEALEALAA (SEQ ID NO:59), Pas, FFLIPKG (SEQ ID NO:60), THRPPMWSPWVWP (SEQ ID NO:61), Angiopep2, TFFYGGSRGKRNNFKTEEY (SEQ ID NO:62), Glutathione, (yE) CG, CDX, FKESWREARGTRIERG (SEQ ID NO:63), Chlorotoxin, MCMPCFTTDHQMARKCDDCCGGKGRGKCYGPQCLCR (SEQ ID NO:64), MiniAP-4, c (DLATEPAL [Dap]) (SEQ ID NO:65), g7, GFTGFLS (Glucose) (SEQ ID NO:66), RV29, YTIWMPENPRPGTPCDIFTNSRGKRASNG (SEQ ID NO:67), iRGD, CRGDKRGPDEC (SEQ ID NO:68), IL-13p, TAMRAVDKLLLHLKKLFREGQFNRNFESIIICRDRT (SEQ ID NO: 69), CGEMGWVRC (SEQ ID NO:70), Lyp-1, c (CGNKRTRGC) (SEQ ID NO:25), DOPAC-MYIEALDKYAC-COOH (SEQ ID NO:71), Pro-Lys-Lys-Lys-Arg-Lys-Val (SEQ ID NO: 72), Ala-Ala-Phe-Glu-Asp-Leu-Arg-Val-Leu-Ser (SEQ ID NO:73), and Lys-Arg-Pro-Ala-Ala-Thr-Lys-Lys-Arg-Gly-Qln-Arg-Lys-Lys-Lys-Lys (SEQ ID NO:74); (xviii) MMP targeting peptides, such as GPLGIAGQ (SEQ ID NO:75); (xix) Transferrin receptor targeting peptides, such as THRPPMWSPVWP (SEQ ID NO:76); (xx) synovial targeting peptides, such as SFHQFARATLAS (SEQ ID NO:77); (xxi) tumor-associated macrophage (TAMs) targeting peptides, such as YEQDPWGVKWWY (SEQ ID NO:78), CSPGAKVRC (SEQ ID NO:79); (xxii) regulatory T lymphocytes (Tregs) targeting peptides, such as CGNKRTRGC (SEQ ID NO: 25); (xxiii) myeloid-derived suppressor cells (MDSCs) targeting peptides, such as MEWSLEKGYTIK (SEQ ID NO:80).

In any aspect or embodiment described herein, T in Formulas IV, V, VI, and is the biologically active molecule (e.g., an agent, such as a therapeutic agent), and the biologically active molecule is covalently linked to the JBNT. Non-limiting examples of the covalently linked biologically active agent include nucleic acids, proteins, peptides, and small molecule drugs. In any aspect or embodiment described herein, the biologically active agent is a nucleic acid such as mRNA, guide RNA (gRNA or sgRNA), crRNA, tracrRNA, IRNA, ssDNA, dsDNA, cDNA, or a combination thereof.

In any aspect or embodiment described herein, the self-assembled nanomaterial includes a biologically active molecule (e.g., an agent, such as a therapeutic agent) that is non-covalently adhered (linked) to the JBNT, and at least partially encapsulated by the JBNT. In any aspect or embodiment described herein, non-limiting examples of the non-covalently linked biologically active agent include nucleic acids, proteins, peptides, and small molecule drugs. In any aspect or embodiment described herein, the biologically active agent is a nucleic acid such as mRNA, guide RNA (gRNA or sgRNA), crRNA, tracrRNA, IRNA, ssDNA, dsDNA, cDNA, or a combination thereof.

In any aspect or embodiment described herein, R² is the coating/protecting material and is selected from polyethylene glycol, chitosan, hyaluronic acid, a poloxamer, polyvinyl alcohol, a polysaccharide, a neutral or negatively charged poly(amino acid), phytochelatin, a self-peptide, an antithrombotic peptide, or a combination thereof. In any aspect or embodiment described herein, R² is a coating/protecting material comprising a polymer, a peptide, a polypeptide, a lipid-based material, phosphate ester, a biomimetic membrane, or a combination thereof. The coating/protecting material may help protect the self-assembled nanomaterial from specific or non-specific clearance from the body by cells and/or organs. In any aspect or embodiment described herein, non-limiting examples of the polymer coating/protecting material include PEG (polyethylene glycol), chitosan, hyaluronic acid, a poloxamer, polyvinyl alcohol, a polysaccharide, a neutral or negatively charged poly(amino acid), or a combination thereof. In any aspect or embodiment described herein, non-limiting examples of the peptide coating/protecting material include a “self” peptide (peptide generated by proteolytic degradation of self protein within cells expressing MHC class I or II molecules, e.g., a TCR-peptide-MHC Class II peptide), an antithrombotic peptide (e.g., CD-31 agonist peptide), CALNN (SEQ ID NO:81), CCVVT (SEQ ID NO:82), CLPFFD (SEQ ID NO:83), (YE) C (yE) C (yE) CG (SEQ ID NO:84), GCGGCGGKGGCGGCG (SEQ ID NO:85), GNYTCEVTELTREGETIIELK (SEQ ID NO:86), hexahistidine, or a combination thereof. Non-limiting examples of the polypeptide (protein) coating/protecting material include phytochelatin, GCK15, PD-L1, CD47, CD24, beta-2-microglobulin, bovine serum albumin (BSA), hydrophobin, clusterin/ApoJ, fibrinogen, or a combination thereof. In any aspect or embodiment described herein, non-limiting examples of the lipid-based coating/protecting material include natural waxes (e.g., carnauba wax, candelilla wax, rice bran wax, beeswax), petroleum based waxes (e.g., paraffin and polyethylene wax), petroleum-based oil, mineral oil, vegetable oil, acetoglycerides, fatty acids, resins (e.g., shellac and wood rosin), or a combination thereof. Non-limiting examples of a biomimetic membrane coating/protecting material include the membranes of red blood cells (RBC), white blood cells (WBC), cancer cells, mesenchymal stem cells, platelets, beta cells, or a combination thereof. A combination comprising at least one of the foregoing coating/protecting materials may also be used.

The self-assembled nanomaterial can include a single type of JBNT or can include more than one JBNT. In an embodiment, the self-assembled nanomaterial is co-assembled from more than one (e.g., a plurality) JBNT, each having properties different from one another. For example, in any aspect or embodiment described herein, the different types of JBNT can have properties which increase the hydrophobicity, stability, and/or self-assembly of the self-assembled nanomaterial. The properties may be designed to affect the cellular delivery, including proton sponge effect, circulation time in vivo, passive or active targeting, subcellular targeting, improved cellular uptake, and/or enhanced endosomal escape of the self-assembled nanomaterial.

In any aspect or embodiment described herein, the total amount of JBNT present in the self-assembled nanomaterial ranges from 0.1 weight % (wt %) to 99.9 wt %, or from 1 wt % to 90 wt %, based on the total weight of the self-assembled nanomaterial. In any aspect or embodiment described herein, the total concentration of the JBNT in the self-assembled nanomaterial ranges from 1 microgram per milliliter (μg/mL) to 1 gram per milliliter (g/mL).

In any aspect or embodiment described herein, the self-assembled nanomaterial is in the form of fibrils, having an average diameter of 50 nm to 2 mm, and an average length of 100 nm to 100 mm.

In any aspect or embodiment described herein, the pH of the self-assembled nanomaterial is from 1 to 10.

In any aspect or embodiment described herein, the self-assembled nanomaterial can have a single or multiple compartment structure. As used herein, in any aspect or embodiment described herein, a single compartment nanomaterial includes a single population of self-assembled nanomaterials, that is, a single type of JBNT and one or more ECM molecules adhering to the JBNT. As used herein, in any aspect or embodiment described herein, a multiple compartment nanomaterial includes two or more populations of self-assembled nanomaterials that form a multi-compartmental structure, through electrostatic layer-by-layer assembly. For example, in any aspect or embodiment described herein, opposite electrostatic charges on the first and second populations of JBNTs can drive assembly of the multiple compartment nanomaterial. By assembling the first and second populations in order, one population will form the interior compartment and the second population will form the exterior compartment.

In any aspect or embodiment described herein, the self-assembled nanomaterial can further include an extracellular matrix (ECM) molecule. In any aspect or embodiment described herein, the self-assembled nanomaterials described herein are tunable materials comprising the Janus base nanotube and the ECM. I any aspect or embodiment described herein, the JBNT can assemble with different ECMs to form different nanomatrix materials, e.g., scaffold, for different cells/tissues. In any aspect or embodiment described herein, these nanomatrix materials can have a single or multiple compartment structure. In addition, the nanomatrix materials can be fabricated with multi-functional layers or compartments to achieve various functions (e.g., supporting cell growth, drug release). In any aspect or embodiment described herein, examples of the ECM molecules include hydroxyapatite, fibronectin, Matn1, MAtn3, laminin, a collagen (e.g., type I collagen, type II collagen), elastin, vitronectin, fibrillin, perlecan, fibrinogen, osteonectin, tenascin, thrombospondin, an intercellular adhesion molecule (ICAM1-5), an integrin, a protcoglycan (aggrecan, a glycosaminoglycan (e.g., hyaluronic acid, chondroitin sulfate, dermatin sulfate, keratan sulfate, heparin, heparin sulfate), a glycoprotein, and combinations thereof.

While not limited thereto, in any aspect or embodiment described herein, the ratio of JBNTs to ECM molecules is 1000:1 to 1:1.

### Self-Assembled Janus Base Nanopieces (Jbnp) for In Vitro Delivery

In any aspect or embodiment described herein, the bioactive self-assembled nanomaterials disclosed herein can be formulated as a self-assembled nanopiece or Janus Base Nanopieces (JBNP) for cellular delivery of an agent (e.g., a therapeutic agent). For example, an aspect of the present disclosure provides a self-assembled nanopiece or Janus Base Nanopiece (JBNP) comprising the self-assembled (bioactive) nanomaterial of the present disclosure and an agent (e.g., therapeutic agent), wherein the self-assembled nanopiece or JBNP optionally comprises, or has attached thereto, a targeting molecule or moiety. For example, in any aspect or embodiment described herein, the agent or therapeutic agent is or includes a nucleic acid vector (e.g., a gene delivery agent (such as, DNA, plasmid, siRNA, miRNA, shRNA)), a protein, a peptide, or a small molecule. In any aspect or embodiment described herein, the self-assembled nanopiece or JBNP further includes a pharmaceutically acceptable carrier (e.g., a pharmaceutically acceptable carrier for gene delivery (such as DNA, plasmid, siRNA, miRNA, shRNA), protein delivery, peptide delivery, or small molecule delivery).

Thus, an aspect of the present disclosure relates to a method of delivering an agent (e.g. a therapeutic agent) to a cell, the method comprising contacting the cell with an effective amount of JBNP of the present disclosure.

In any aspect or embodiment described herein, the agent or therapeutic agent is or includes a gene editing reagent (e.g., DNA editing, RNA editing, or both). For example, in any aspect or embodiment described herein, the agent or therapeutic agent includes gene editing related-mRNA (e.g., Cas9 mRNA, dCas9 mRNA, APOBEC mRNA, gRNA, tracrRNA, crRNA, SSDNA, cDNA, dsDNA, or a combination thereof).

In any aspect or embodiment described herein, the JBNP comprises a peptide/protein (JBNP-peptide). In any aspect or embodiment described herein, the JBNP-peptide is prepared by mixing the appropriate ratio between the peptides and the JBNTs, and after mixing, the mixture is sonicated with Sonicator (Q, Sonica; Sonicators) at properly designed amplitude for 2 minutes and 30 seconds. In any aspect or embodiment described herein, the sonication amplitude for JBNP comprising peptide/protein is about 30% to 50% (e.g., about 30% to about 50%, about 30% to about 40%, or about 40% to about 50%).

In any aspect or embodiment described herein, the JBNP comprises a targeting peptide (targeting peptide-JBNP), which can be used to deliver the peptide to one or more cells. For example, in any aspect or embodiment described herein, the assembled targeting-peptide-JBNP comprises Interleukin 1-alpha (JBNP-IL-1A or IL-1A-JBNP). For example, in any aspect or embodiment described herein, the IL-1A has the amino acid sequence: MRPSGRKSSKMQAFRIWDVNQKTFYLRNNQLVAGYLQGPNVNLEEKIDVVPIEPHALF LGIHGGKMCLSCVKSGDETRLQLEAVNITDLSENRKQDKRFAFIRSDSGPTTSFESAACP GWFLCTAMEADQPVSLTNMPDEGVMVTKFYFQEDE (SEQ ID NO:87). In any aspect or embodiment described herein, the JBNP-IL-1A is administered, delivered, or contacted withone or more cells (e.g., human chondrocytes (e.g. C28/12)), thereby reducing or suppressing the expression of one or more inflammatory genes (such as matrix metalloproteinase 13 (MMP-13) and A disintegrin and metalloproteinase with thrombospondin motifs 5 (ADAMTS-5)).

In any aspect or embodiment described herein, reverse transcriptase-quantitative polymerase chain reaction (RT-qPCR) can be utilized to measure the level of MMP-13, ADAMTS-5, or both. In any aspect or embodiment described herein, western blot can be utilized to measure the level of MMP-13 and ADATS-5.

In any aspect or embodiment described herein, the assembled JBNP comprises Interleukin-1 receptor antagonist (JBNP-IL-1RA). For example, in any aspect or embodiment described herein, the IL-1RA has the amino acid sequence: MEICRGLRSHLITLLLFLFHSETICRPSGRKSSKMQAFRIWDVNQKTFYLRNNQLVAGYL QGPNVNLEEKIDVVPIEPHALFLGIHGGKMCLSCVKSGDETRLQLEAVNITDLSENRKQ DKRFAFIRSDSGPTTSFESAACPGWFLCTAMEADQPVSLTNMPDEGVMVTKFYFQEDE (SEQ ID NO:88). In any aspect or embodiment described herein, the JBNP-IL-1RA is administered, delivered, or contacted with one or more cells (e.g., human chondrocytes (e.g., C28/12)), thereby reducing or suppresses the expression of one or more inflammatory genes (such as MMP-13 and ADAMTS-5).

In any aspect or embodiment described herein, the JBNP is prepared by mixing an appropriate ratio between the agent or therapeutic agent and the JBNT of the present disclosure. In any aspect or embodiment described herein, the proper ratio is determined by the ratio of positively-chargeable polymer amine (N=nitrogen) groups to negatively charged nucleic acid phosphate (P) groups, N/P ratio. After mixing the JBNT and agent or therapeutic agent at the desired N/P ratio, the mixture is sonicated with Sonicator (Q, Sonica; Sonicators) at properly designed amplitude for 2 minutes and 30 seconds.

In any aspect or embodiment described herein, the N/P ratio is determined experimentally and can optionally include machine-learning base regression. In any aspect or embodiment described herein, the N/P ratio is further selected to deliver to cells based on the characteristics of the JBNP. In any aspect or embodiment described herein, the characteristics of the JBNP utilized to determine the N/P ratio including surface charge, hydrodynamic size, ultraviolet visible (UV-VIS) spectroscopy characteristics, or a combination thereof.

In any aspect or embodiment described herein, the JBNP comprises DNA (JBNP-DNA). In any aspect or embodiment described herein, the JBNP-DNA is prepared by mixing the appropriate ratio between the JBNTs and the DNA, and after mixing, the mixture is sonicated with Sonicator (Q, Sonica; Sonicators) at properly designed amplitude for 2 minutes and 30 seconds. In any aspect or embodiment described herein, the sonication amplitude for JBNP comprising DNA is about 50% to about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 700% to about 100%, about 70% to about 90%, about 70% to about 80%, about 80% to about 100%, about 80% to about 90%, or about 90% to about 100%).

In any aspect or embodiment described herein, the JBNP comprises mRNA (JBNP-mRNA). In any aspect or embodiment described herein, the JBNP-mRNA has a positive surface charge of about 10 to about 30 mV (e.g., about 10 to about 30 mV, about 10 to about 25 mV, about 10 to about 20 mV, about 10 to about 15 mV, about 15 to about 30 mV, about 15 to about 25 mV, about 15 to about 20 mV, about 20 to about 30 mV, about 20 to about 25 mV, or about 25 to about 30 mV), about 5 to about 300 nm (e.g., about 50 to about 300 nm, about 50 to about 250 nm, about 50 to about 200 nm, about 50 to about 150 nm, about 50 to about 100 nm, about 100 to about 300 nm, about 100 to about 250 nm, about 100 to about 200 nm, about 100 to about 150 nm, about 150 to about 300 nm, about 150 to about 250 nm, about 150 to about 200 nm, about 200 to about 300 nm, about 200 to about 250 nm, or about 250 to about 300 nm) of hydrodynamic size, or a combination thereof. In any aspect or embodiment described herein, assembled JBNP-mRNA (such as with different N/P ratio) is characterized by their hydrodynamic size using the Dynamic Light Scattering (DLS), surface charge by Zetasizer (Malvern Panalytical), or a combination thereof. In any aspect or embodiment described herein, the JBNP-mRNA is prepared by mixing the appropriate ratio between the mRNA and the JBNTs, and after mixing, the mixture is sonicated with Sonicator (Q, Sonica; Sonicators) at properly designed amplitude for 2 minutes and 30 seconds. In any aspect or embodiment described herein, sonication amplitude for JBNP comprising mRNA is about 20%.

In any aspect or embodiment described herein, the JBNP-mRNA has an N/P ratio of about 121 to about 350 (e.g., about 121 to about 350, about 121 to about 300, about 121 to about 250, about 121 to about 200, about 150 to about 350, about 150 to about 300, about 150 to about 250, about 150 to about 200,about 200 to about 350, about 200 to about 300, about 200 to about 250, about 250 to about 350, about 250 to about 300, or about 300 to about 350), which is effective to deliver the mRNA to a cell. For example, the delivery of JBNP-mRNA can be examined via the following method. Human chondrocytes (50000 cells/ml) are seeded in a 4 well-confocal well overnight. Then, JBNP-mcherry mRNA is transfected to human chondrocytes, C28/12 cells for 72 hours. After 72 hours, a confocal laser-scanning microscope can be used for fluorescence imaging. To quantify, 50000 cells/well of human chondrocytes can be seeded into 24 wells overnight. Then, JBNP-mcherry mRNA is transferred to the wells. After 72 hours, fluorescence-activated cell sorting (FACS) quantifies the mCherry signal. To demonstrate the mRNA translation to protein, mCherry translation expression is analyzed by western-blot.

In any aspect or embodiment described herein, the JBNP comprises siRNA (JBNP-siRNA). In any aspect or embodiment described herein, the JBNP-siRNA has an N/P ratio is about 40 to about 100 (e.g., about 40 to about 100, about 40 to about 85, about 40 to about 70, about 40 to about 55, about 50 to about 100, about 50 to about 85, about 50 to about 70, about 60 to about 100, about 60 to about 85, about 60 to about 70, about 70 to about 100, about 70 to about 85, or about 80 to about 100). For example, the delivery of JBNP-siRNA can be examined via the following method. Administer assembled JBNP-siRNA-fluorescent tag (e.g. AF488) to one or more cell (e.g., C28/12 cells). After a 24 hour incubation, the cells can be examined for fluorescence (e.g., delivery efficiency examined/determined, mean fluorescence intensity examined/determined, or both), such as for AF488, via fluorescence imaging (e.g., with a confocal laser-scanning microscope), FACS, or both. By way of further example, siRNA knockdown can be examined using the following method. JBNP-siRNA (e.g., glyceraldehyde 3-phosphate dehydrogenase (GAPDH) siRNA) is administered to one or more cells. After a 24 hour incubation, the reduced expression by the administration of the siRNA can be analyzed by RT-qPCR for the siRNA target (e.g., GAPDH), western-blot for the siRNA (e.g., GAPDH), or both. In any aspect or embodiment described herein, the JBNP-siRNA is prepared by mixing the appropriate ratio between the JBNTs and the siRNA, and after mixing, the mixture is sonicated with Sonicator (Q, Sonica; Sonicators) at properly designed amplitude for 2 minutes and 30 seconds. In any aspect or embodiment described herein, the sonication amplitude for JBNP comprising siRNA is about 50% to about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 700% to about 100%, about 70% to about 90%, about 70% to about 80%, about 80% to about 100%, about 80% to about 90%, or about 90% to about 100%).

In any aspect or embodiment described herein, the JBNP comprises a small molecule (JBNP-small molecule). In any aspect or embodiment described herein, the JBNP-small molecule is prepared by mixing the appropriate ratio between the JBNTs and the small molecule, and after mixing, the mixture is sonicated with Sonicator (Q, Sonica; Sonicators) at properly designed amplitude for 2 minutes and 30 seconds. In any aspect or embodiment described herein, the sonication amplitude for JBNP comprising a small molecule is about 50% to about 100% (e.g., about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 700% to about 100%, about 70% to about 90%, about 70% to about 80%, about 80% to about 100%, about 80% to about 90%, or about 90% to about 100%). For example, in any aspect or embodiment described herein, a JBNP-small molecule has a working N/P ratio of about 15 to about 20 (e.g., about 15, about 16, about 17, about 18, about 19, or about 20). In any aspect or embodiment described herein, the JBNP-small molecule is or comprises an anticancer drug (e.g., doxorubicin (JBNP-DOXs)). In any aspect or embodiment described herein, the delivery of JBNP-small molecule, such as assembled JBNP-DOXs can be examined via the following method. JBNP-DOXs is administered to human ovarian cancer cell (SKOV-3) cells. After a 48 hour incubation, a confocal laser-scanning microscope can be used to image the small molecule (e.g., doxorubicin (DOX)), FACS utilized to quantify delivery efficiency of the small molecule (e.g., DOX), or both. Furthermore, when the small molecule of the JBNP-small molecule is an anticancer drug, an apoptosis assay can be performed/utilized to examine the anti-cancer effect of the JBNP-small molecule administration.

In any aspect or embodiment described herein, the dosage of the agent or therapeutic agent of the JBNP is determined experimentally and extends to machine-learning base regression approach. Example, in any aspect or embodiment described herein, the dosage of the mRNA of a JBNP-mRNA is about 0.025 ug to about 0.05 ug (e.g., about 0.025 ug to about 0.05 ug, about 0.025 ug to about 0.04 ug, about 0.025 ug to about 0.03 ug, about 0.03 ug to about 0.05 ug, about 0.03 ug to about 0.04 ug, or about 0.04 ug to about 0.05 ug,). By way of further example, in any aspect or embodiment described herein, the dosage of the siRNA of a JBNP-siRNA is about 0.05 ug to about 0.1 ug (e.g., about 0.05 ug to about 0.1 ug, about 0.05 ug to about 0.09 ug, about 0.05 ug to about 0.08 ug, about 0.05 ug to about 0.07 ug, about 0.05 ug to about 0.06 ug, about 0.06 ug to about 0.1 ug, about 0.06 ug to about 0.09 ug, about 0.06 ug to about 0.08 ug, about 0.06 ug to about 0.07 ug, about 0.07 ug to about 0.1 ug, about 0.07 ug to about 0.09 ug, about 0.07 ug to about 0.08 ug, about 0.08 ug to about 0.1 ug, about 0.08 ug to about 0.09 ug, or about 0.09 ug to about 0.1 ug). Furthermore, in any aspect or embodiment described herein, the dosage of the small molecule of the JBNP-small molecules is about 0.2 ug to about 0.3 ug.

In any aspect or embodiment described herein, the apparent pKa of JBNP is about 6 to about 7, which is the optimum range for the JBNP to deliver RNAs into a cell.

It was discovered that the JBNP of the present disclosure have enhanced endosomal escape as compared to conventional nanoparticles, such as lipid nanoparticles. Furthermore, based on the JBNT modification, JBNP shows enhanced endosomal escape. For example, JBNP comprising lysine-based JBNT have significantly better endosomal escape, likely due to the proton sponge effect. By way of further example, JBNP comprising arginine-based JBNT have significantly enhanced endosomal escape, likely due to pore-forming effect to escape early endosome.

In any aspect or embodiment described herein, the apparent pKa of the JBNP of the present disclosure is determined by 2-(p-toluidino)-6-napthalene sulfonic acid (TNS) assay. For example, in any aspect or embodiment described herein, the JBNP-mRNA formulation and the TNS probe (2 uM) are incubated in a black-bottom 96-well plate for 5 minutes with a series of buffers (20 mM citrate buffer (pH 3.50-5.50), 20 mM sodium phosphate buffer (pH 6.00-8.00), or 20 mM Tris-HCl buffer (pH 8.00-9.00)), and the mean fluorescence intensity of each well of the plate measured by a plate reader.

In any aspect or embodiment described herein, the JBNP is an extracellular matrices (ECM)-targeting JBNP (e.g., iRGD-JBNP) comprising, or that is attached thereto, a extracellular matrices targeting molecule or moiety (e.g., iRGD) can interact with ECMs and alters cellular signaling. In any aspect or embodiment described herein, the bioactive JBNP materials are engineered to increase the therapeutic cellular signaling (such as enhanced neuronal signal for brain disease). In any aspect or embodiment described herein, the change in cellular signaling can be examined by delivering/contacting assembled extracellular matrices-targeting JBNP (e.g., iRGD-JBNP) with siRNA-AF488 to cells (e.g., C28/12 cells), and after a 24 hour incubation, performing RNA sequencing (RNA-seq) to detect changes in the cellular signaling of the cell and to quantify the mRNA in a single cell.

In any aspect or embodiment described herein, the JBNP is used to perform gene editing. For example, in any aspect or embodiment described herein, the JBNP comprises Cas9mRNA and guide RNA (gRNA or sgRNA) (JBNP-Cas9mRNA-gRNA or JBNP-CRISPR), which can contacted, delivered, or administered to one or more cells.

In any aspect or embodiment described herein, Cas9mRNA and gRNA of the JBNP-Cas9mRNA-gRNA or JBNP-CRISPR are delivered with an optimal ratio of w/w of Cas9 mRNA:gRNA. In any aspect or embodiment described herein, the optimal dosage of JBNP-CRISPR is contacted, delivered, or administered to the cell. In any aspect or embodiment described herein, the optimal ratio and dosage is determined by machine-learning base regression. In any aspect or embodiment described herein, cells are contacted, delivered, or administered to one or more cells. For example, the following method can be utilized to examine the JBNP-CRISPR. For example, cells (such as RFP Expressing Human Umbilical Vein Endothelial Cells (RFP-HUVECs)) are seeded into a plate (e.g., a 24-well plate with 50000 cells/well) and incubated overnight. The cells with RFP (e.g., RFP-HUVECs) are transfected with JBNP-CRISPR comprising Cas9mRNA and sgRFP for 72 hours. After the incubation, the mean fluorescence intensity was measured by FACS. In any aspect or embodiment described herein, T7 endonuclease 1 (T7E1) assay is used to determine gene editing efficiency of the JBNP-CRISPR. In any aspect or embodiment described herein, Sanger sequencing is used to determine the editing efficiency of the JBNP-CRISPR. In any aspect or embodiment described herein, off-target analysis for the JBNP-CRISPR is performed with GUIDE-seq, BLISS, CIRCLE-seq, DISCOVER-Seq, LAM PCR HTCTS, Digenome-seq, or a combination thereof. In any aspect or embodiment described herein, SITE-seq is used to examine genotoxicity of the JBNP-CRISPR.

In any aspect or embodiment described herein, the JBNP comprises, or has attached thereto, a targeting molecule or moiety. For example, in any aspect or embodiment described herein, the targeting molecule or moiety targets the JBNP to one or more subcellular compartment (e.g., nucleus, endoplasmic reticulum, mitochondria, golgi apparatus, or a combination thereof).

In any aspect or embodiment described herein, targeting can be optimized by using varying ratios of targeting molecule/moiety-JBNT to non-targeting-JBNT (i.e., JBNT without a targeting molecule/moiety). In any aspect or embodiment described herein, the ratio of targeting molecule/moiety-JBNT to non-targeting-JBNT is about 1:100 to about 1:20 for golgi apparatus targeting, about 1:20 to 1:10 for mitochondrial targeting, about 1:10 to about 2:5 for endoplasmic reticulum targeting. In any aspect or embodiment described herein, the following method can be utilized to determine the localization of the JBNP with a targeting molecule or moiety for one or more subcellular compartment of a cell (subcellular compartment targeting-JBNP). For example, assembled subcellular compartment targeting-JBNP (e.g., targeting-JBNP-AF488) is immediately transferred to cultured cells (e.g., C28/12 cells) and incubated for 24 hours. Then, the cells were fixed with 4% formaldehyde, and immunohistochemistry (IHC) staining performed for one or more desired subcellular compartments (e.g., a stain can be used for golgi apparatus). Co-localization of the subcellular compartment targeting-JBNP and the one or more stained subcellular compartment(s) can then be achieved (e.g., the co-localization can be quantified with Pearson's R value).

In any aspect or embodiment described herein, the ratio of targeting molecule/moiety-JBNT to non-targeting-JBNT is about 1:100 to about 1:10 for targeting a subcellular compartment. In any aspect or embodiment described herein, the ratio of targeting molecule/moiety-JBNT to non-targeting-JBNT is about 1:10 to about 1:2 for targeting an organ.

In any aspect or embodiment described herein, the JBNP achieves longer circulation time and reduces the nonspecific interaction by attaching R² to PEG, as described herein. In any aspect or embodiment described herein, the circulation time can be modified, or tailored to a desired period of time, by mixing based on the ratio between PEG-JBNT and non-PEG-JBNT. For in aspect or embodiment described herein, wherein a longer circulation time is desired, an optimized ratio of about 30% to about 50% of the mixture of JBNT and PEG-JBNT is PEG-JBNT. In any aspect or embodiment described herein, the circulation of the can be examined in vitro or in vivo with JBNP-mRNA-Cy5s. For example, the time-dependent fluorescence signal from mRNA-cy5 (e.g., at one or more of 1 hour, 3 hours, 6 hours, 12 hours, 24 hours, 48 hours, 72 hours) of JBNP-mRNA-Cy5 in the serum is measured.

In any aspect or embodiment described herein, the self-assembled nanomaterials may be administered parenterally in a sterile medium, either subcutaneously, or intravenously, or intramuscularly, or intrasternally, or by infusion techniques, in the form of sterile injectable aqueous or oleaginous suspensions. In any aspect or embodiment described herein, advantageously, adjuvants such as a local anaesthetic, preservative, and buffering agents can be dissolved in the vehicle.

In any aspect or embodiment described herein, sodium dodecyl-sulfate polyacrylamide gel electrophoresis (SDS-PAGE) is utilized/performed to visualize or examine the protein or proteins attached to the JBNP. In any aspect or embodiment described herein, liquid chromatography tandem mass spectroscopy (LC-MS/MS) is utilized/performed to analyze the protein bands on the SDS-PAGE gel (e.g., quantify the composition).

### Self-Assembled Janus Base Nanopieces (Jbnp) for In Vivo Delivery

In any aspect or embodiment described herein, the self-assembled bioactive nanomaterials of the present disclosure can be formulated as a self-assembled nanopiece or Janus Base nanopieces (JBNP) for in vivo deliver of an agent (e.g., a therapeutic agent), wherein the JBNP optionally comprises, or has attached thereto, a targeting molecule or moiety. For example, in any aspect or embodiment described herein, the method comprising administering an effective amount of the self-assembled nanopiece or JBNP of the present disclosure to a subject (e.g., a patient or a human). Furthermore, an aspect of the present disclosure relates to a method of treating, preventing, or ameliorating one or more symptoms of, a disease, disorder, or condition in a subject, the method comprising administering an effective amount of the self-assembled nanopiece or JBNP of the present disclosure to the subject (e.g., a patient or a human).

In any aspect or embodiment described herein, an organ targeting molecule or moiety that targets an organ is attached to the self-assembled nanopiece or JBNP. For example, the organ targeting molecule or moiety can specifically target the self-assembled nanopiece or JBNP to the brain, spleen, pancreas, lung, liver, heart, bone, lymph nodes, muscle, kidney, or a combination thereof. Thus, in any aspect or embodiment described herein, a JBNP with organ-targeting molecule or moiety can achieve active targeting to one or more organs. In any aspect or embodiment described herein, the targeting molecule or moiety includes a peptide, cyclic peptide, small molecule, another molecular structure mentioned herein as a targeting molecule or moiety, or a combination thereof.

In any aspect or embodiment described herein, a tumor targeting molecule or moiety that targets a tumor is attached to the JBNP. In any aspect or embodiment described herein, the tumor targeting molecule or moiety targets the JBNP to one or more tumor when systematically delivery, intra-tumorally delivery, or both.

In any aspect or embodiment described herein, a fluorescent molecule (e.g., Cy5) is attached to the agent or therapeutic agent (e.g., mRNA-Cy5). For example, in any aspect or embodiment described herein, the JBNP comprising an agent or therapeutic agent with a fluorescent molecule attached thereto is systemically administered to a subject (e.g., a mouse, rat, rabbit, etc.), and the fluorescence intensity is measured and biodistribution examined via IVIS® imaging. For example, in any aspect or embodiment described herein, the biodistribution is examined at about 12 hours, about 24 hours, about 36 hours, about 48 hours, about 60 hours, about 72 hours, about 84 hours, or a combination thereof, post administration. For example, biodistribution can be examined in BALB/cJ mice (e.g., 12 mice) after tail-vein injection with the JBNP comprising an agent or therapeutic agent with a fluorescent molecule attached thereto (e.g., JBNP-mRNACy5) or saline as a negative control.

In any aspect or embodiment described herein, the JBNP comprises, or has attached thereto, reporter mRNA (e.g., Fluc mRNA). For example, in any aspect or embodiment described herein, the reporter mRNA is attached to the agent or therapeutic agent (e.g., mRNA, thereby forming JBNP-mRNA-Fluc mRNA). In any aspect or embodiment described herein, the JBNP comprising reporting mRNA is systematically administered (e.g., intravenously (i.v.) administered) to a subject (e.g., a mouse, rat, rabbit, etc.), luciferin is injected (e.g., intraperitoneal (i.p.) injection), and fluorescent intensity and biodistribution is measured via IVIS® imaging. For example, in any aspect or embodiment described herein, the biodistribution is examined at about 12 hours, about 24 hours, about 36 hours, about 48 hours, about 60 hours, about 72 hours, about 84 hours, or a combination thereof, post administration. For example, in any aspect or embodiment described herein, the luciferin is injected about 8 to about 20 minutes (e.g., about 8, about 9, about 10, about 11, about 12, about 13, about 14, about 15, about 16, about 17, about 18, about 19, or about 20 minutes) before measuring fluorescent intensity and examining biodistribution. For example, biodistribution is examined in BALB/cJ mice (e.g., 12 mice) after tail-vein injection with the JBPN comprising an agent or therapeutic agent (such as mRNA) with an mRNA reporter attached thereto (e.g., JBNP-mRNA-Fluc mRNA) or saline as a negative control. In any aspect or embodiment described herein, the dosage and/or delivery time can be examined to optimize the biodistribution.

In any aspect or embodiment described herein, the JBNP comprises, or has attached thereto, a bone targeting molecule or moiety to facilitate the delivery of the agent or therapeutic agent to bone, which may be used to treat a bone. For example, in any aspect or embodiment described herein, the bone-targeting JBNP is a bisphosphonates-JBNP (e.g., bisphosphonates-JBNP-mRNA or bisphosphonates-JBNP-mRNA-Cy5). In any aspect or embodiment described herein, bone and bone marrow transfection can be examined via the following method. For example, assembled bisphosphonates-JBNP can target the bone systemically. For example, bisphosphonates-JBNP-mRNA-Cy5 administered intravenously to mice (e.g., BALB/cJ mice) for 24 hours, and biodistribution is monitored with IVIS® imaging. Cell transfection of and distribution in bone and/or bone marrow cells, in any aspect or embodiment described herein, can be examined and/or quantified by homogenizing bone and performing FACS.

In any aspect or embodiment described herein, the JBNP comprises, or has attached thereto, a blood-brain barrier targeting molecule or moiety (e.g., transferrin) that facilitates the delivery of the agent or therapeutic agent to, and penetrates, the blood-brain barrier, which may be used in a method to treat a brain. For example, in any aspect or embodiment described herein, the blood-brain barrier targeting-JBNP is transferring-JGNP-siRNA, optionally with a fluorescent marker attached thereto (e.g., AF488). In any aspect or embodiment described herein, delivery to and the ability to penetrate the blood-brain barrier of a JBNP (e.g., transferrin-JBNP or transferrin-JBNP-siRNA, each optionally including a marker, such as a fluorescent market, such as AF488) can be examined using the transwell in vitro brood-brain barrier model. For example, transfecting the transwell (blood-brain barrier composed of hcMEC/D3 cells) with transferrin-JBNP-AF488 (e.g., transferrin-JBNP-siRNA-AF488) for 6 hours. Then, the AF488 (e.g., siRNA-AF488) signal is measured on hcMEC/D3 to quantify the degree of penetration. In any aspect or embodiment described herein, delivery to and the ability to penetrate the blood-brain barrier of JBNP (e.g., transferrin-JBNP or transferrin-JBNP-siRNA, each optionally including a marker, such as AF647) can be examined using mice and IVIS® imaging. For example, intravenously injecting assembled transferrin-JBNP-AF647 (e.g., transferrin-JBNP-siRNA-AF647) into mice (e.g., BALB/cJ mice or 5XFAD micc) for 12 hours, and measuring fluorescence intensity on the brain using IVIS® imaging.

In any aspect or embodiment described herein, the JBNP comprises, or has attached thereto, a blood-brain barrier targeting molecule or moiety (e.g., transferrin) that facilitates the delivery of the agent or therapeutic agent to, and penetrates, the blood-brain barrier, which may be used in a method to treat a brain, and the agent or therapeutic agent is an siRNA that targets beta-secretasel (BACE1) (BAC1 siRNA). In any aspect or embodiment described herein, the blood-brain barrier targeting JBNP-BACE1 siRNA (e.g., transferrin-JBNP-BACE1 siRNA) can be evaluated for its ability to affect BACE1 protein expression and treat Alzheimer in 5XFAD mice. For example, assembled transferrin-JBNP-BACE1 siRNA can be intravenously injected (e.g., at different dose strategies) into 5XFAD mice for 90 days (e.g., every 5 days for 90 days). Then, one or more behavioral tests that are used to evaluate Alzheimer's (such as, Y-maze, open field, novel object recognition, fear conditioning, or a combination thereof) can be conducted to examine mice learning and memory. Post 90 days, brain tissues can be collected to analyze the BACE1 gene and/or BACE1 protein expression using the real-time RT-PCR, ELISA, and/or western blot. Furthermore, amyloid plaque can be evaluated using the IHC staining and/or electron microscopy.

In any aspect or embodiment described herein, the JBNP passively targets a tumor via the enhanced permeability and retention (EPR) effect, and optionally the agent or therapeutic agent is an anticancer drug. For example, in any aspect or embodiment described herein, the agent or therapeutic agent of the JBNP is an anticancer drug, and the JBNP effectively accumulates inside solid tumors, thereby facilitating the delivery of the anticancer drug and inhibiting tumor growth. In any aspect or embodiment described herein, the following method can be used to examine JBNPs ability to accumulate and/or treat tumors. For example, Matrigel® Matrix is mixed with transplantable tumor cells, and a nude mouse (or mice) is inoculated intradermal or subcutaneous with the mixture containing 1×10e8 tumor cells. After the tumor volume reaches 50 mm³, JBNP-anticancer agent (e.g., JBNP-DOX) is injected every 3 days for 3 weeks. Biodistribution of the JBNP-DOX can be examined by PerkinElmer® IVIS® imaging. Furthermore, the mouse/mice can be monitored for tumor size, weight, complete blood count (CBC), tumor necrosis factor-alpha (TNF-alpha) levels, interferon-gamma (IFN-gamma) levels, immunoglobulin G (lgG) levels, immunoglobulin M (IgM) levels, or a combination thereof. The mouse/mice can be further examined/assayed for toxicity.

In any aspect or embodiment described herein, the JBNP comprises, or is attached thereto, a tumor-targeting molecule or moiety (e.g., a tumor targeting peptide, such as KDEPQRRSARLSAKPAPPKPEPKPKKAPAKK, SEQ ID NO:24), which actively target the tumor to deliver the agent or therapeutic agent (e.g., a tumor targeting-JBNP, tumor targeting peptide-JBNP, or KDEPQRRSARLSAKPAPPKPEPKPKKAPAKK-JBNP), and optionally the agent or therapeutic agent is an anticancer drug (e.g., DOX). For example, in any aspect or embodiment described herein, the agent or therapeutic agent of the JBNP is an anticancer drug, and the JBNP effectively accumulates inside solid tumors a tumor-targeting molecule or moiety (e.g., a tumor-targeting peptide, such as KDEPQRRSARLSAKPAPPKPEPKPKKAPAKK, SEQ ID NO: 24), thereby facilitating the delivery of the anticancer drug and inhibiting tumor growth. In any aspect or embodiment described herein, the method can be used to examine JBNPs ability to accumulate and/or treat tumors. For example, Matrigel® Matrix is mixed with transplantable tumor cells, and a nude mouse (or mice) is inoculated intradermal or subcutaneous with the mixture containing 1×10e8 tumor cells. After the tumor volume reaches 50 mm³, the tumor-targeting-JBNP-anticancer drug (e.g., KDEPQRRSARLSAKPAPPKPEPKPKKAPAKK-JBNP-DOX) is injected every 3 days for 3 weeks. Biodistribution of the tumor-targeting-JBNP-anticancer drug can be explored by PerkinElmer® IVIS® imaging. Furthermore, the mouse/mice can be monitored for tumor size, weight, complete blood count (CBC), tumor necrosis factor-alpha (TNF-alpha) levels, interferon-gamma (IFN-gamma) levels, immunoglobulin G (lgG) levels, immunoglobulin M (IgM) levels, or a combination thereof. The mouse/mice can be further examined/assayed for toxicity.

In any aspect or embodiment described herein, the JBNP comprises, or has attached thereto, a spleen targeting molecule or moiety (e.g., a spleen targeting peptide) or an immune cell targeting molecule or moiety (e.g., a immune cell targeting peptide). For example, in any aspect or embodiment described herein, the JBNP-Cas9mRNA-gRNA (JBNP-CRISPR), comprises, or has attached thereto, a spleen or immune cell targeting molecule or moiety (e.g., a spleen or immune cell targeting peptide). In any aspect or embodiment described herein, the spleen or immune cell targeting molecule moiety (e.g., a spleen or immune cell targeting peptide) targets CD4+ T cells, CD8+ T cells, B cells, or a combination thereof. In any aspect or embodiment described herein, the CRISPR specifically edits the DNA of CD4+ T cells, CD8+ T cells, B cells, or a combination thereof. In any aspect or embodiment described herein, the CRISPR is or includes a Cre-Controlled CRISPR (3C), optionally comprising Cre mRNA. In any aspect or embodiment described herein, the following method can be used to examine spleen targeting-JBNPs ability to target immune cells in the spleen and/or lymph nodes. In any aspect or embodiment described herein, the following method can be used to examine of JBNPs with a spleen targeting molecule moiety (spleen targeting-JBNP) or immune cell targeting molecule or moiety (immune cell targeting-JBNP) to effectively target the spleen or immune cells. For example, intravenously inject spleen targeting-JBNP or immune cell targeting-JBNP (e.g., CD4+ T cell targeting-JBNP) comprising an mRNA reporter (e.g., tandem dimer Tomato (tdTomato) RNA) to a mouse (or mice), such as Ai14 mice, and examine the spleen and lymph nodes for expression of the mRNA reporter (e.g., tdTomato) by PerkinElmer® IVIS® imaging. For example, IVIS® imaging can be used to quantify based on the expression of the mRNA reporter. Furthermore, in any aspect or embodiment described herein, FACS can be performed on spleen homogenate and/or lymph node homogenate to quantify delivery efficiency to the spleen, immune cells in the spleen, and/or immune cells in lymph nodes.

In any aspect or embodiment described herein, JBNP is used as a cancer immunotherapy whose anti-tumor effect is due to the subject's (e.g., patient, human, xenograft tumor model, etc.) immune response. In any aspect or embodiment described herein, a xenograft tumor model, such as the xenograft SKOV-3 tumor model, can be used to examine the in vivo efficacy of a JBNP designed for cancer immunotherapy. For example, JBNP designed for cancer immunotherapy is intravenously injected or intra-tumorally injected into the experimental animal of the xenograft tumor model (e.g., a nude mouse/mice), and the anti-tumor effect examined. For example, JBNP-CRISPR designed for cancer immunotherapy is intravenously injected or intra-tumorally injection once into the experimental animal of the xenograft tumor model (e.g., xenograft SKOV-3 tumor model), and the anti-tumor effect examined. For example, in any aspect or embodiment described herein, the efficacy of an anti-tumor immune response (e.g., tumor growth, transcriptomc (such as inflammatory transcriptome) analysis of the tumor with RNA-seq, or both), immune response (e.g., determine the concentration of IL-12 and/or INF-gamma in the serum from peripheral blood), toxicity, or a combination thereof, is examined.

In any aspect or embodiment described herein, the JBNP-CRISPR is designed and/or utilized in a method as described herein, to edit the DNA of cells for ex vivo treatment, such as ex vivo immunotherapy or ex vivo cancer immunotherapy. For example, in any aspect or embodiment described herein, the JBNP-CRISPR is designed to edit the DNA of immune cells, such as β-cells, CD4+ T cells, CD8+ T cells, chimeric antigen receptor T cells (CAR-T cells, such as CD8+ CAR-T-cell, CD4+ CAR-T-cell, or both), and macrophage. In any aspect or embodiment described herein, the JBNP-CRISPR edited immune cells are transfused into a subject (e.g., a mouse), such as the subject from which they were obtained (autologous treatment). Thus, the JBNP-CRISPR edited immune cells can be from the subject to which they are going to be administered (autologous treament) or from a healthy donor (allogeneic treatment).

In any aspect or embodiment described herein, the JBNP-CRISPR is a gene editing platform for CAR-T cell treatment/therapy. For example, in any aspect or embodiment described herein, the JBNP-CRISPR is designed to edit the negative regulators of T-cells, CD4+ T cells, CD8+ T cells, or a combination thereof, thereby improving their anti-tumor effect. In any aspect or embodiment described herein, the following method can be utilized to examine JBNP-CRISPR edited T cells can be prepared via the following method. For example, assembled JBNP-CRISPR designed to edit one or more genes from T-cells, CD4+ T cells, CD8+ T cells, or a combination thereof, is incubated with a cell population comprising the target cells for 72 hours. In any aspect or embodiment described herein, the JBNP-CRISPR gene edited T-cells, CD4+ T cells, and/or CD8+ T cells are selected through FACS, and expanded prior to transfusion. In any aspect or embodiment described herein, JBNP-CRISPR gene edited T-cells, CD4+ T cells, and/or CD8+ T cells (e.g., a 1:1 ratio of CD4+ T cells and CD8+ T cells, such as cells that have one or more genes knocked out) are transfused to an experimental animal of a cancer xenograft model, and the efficacy of an anti-tumor immune response (e.g., tumor growth, transcriptome (such as inflammatory transcriptome) analysis of the tumor with RNA-seq, or both), immune response (e.g., determine the concentration of IL-12 and/or INF-gamma in the serum from peripheral blood), toxicity, or a combination thereof, is examined.

In any aspect or embodiment described herein, the JBNP comprises an agent or therapeutic agent that reduces or prevents inflammation, such as that which is observed osteoarthritis to which it can prevent, treat, or prevent the progression of. In any aspect or embodiment described herein, the effectiveness of a JBNP comprising an agent or therapeutic agent that reduces or prevents inflammation associated with osteoarthritis can be examined via the following method. For example, the medial meniscus (DMM) of a 129SVE-M mouse (or mice) is surgically destabilized, and the mouse monitor for the development the osteoarthritis about 4 to 12 weeks. At the designated time point (e.g., prior to development of osteoarthritis for examination of prevention of or progression of versus after development of osteoarthritis for examination of treatment), an intra-articular injection of the JBNP comprising an agent or therapeutic agent that reduces or prevents inflammation (e.g., JBNP-mRNA-Cy5) into the knee joint is performed, and the mouse monitored for fluorescence via IVIS® imaging. For example, retention examined via IVIS® imaging. Furthermore, in any aspect or embodiment described herein, treatment efficacy can be examined via behavioral studies, such as Y-Maze Spontaneous Alteration, Open Field, Object Recognition Test, Fear Conditioning, or a combination thereof.

In any aspect or embodiment described herein, the JBNP comprises a cartilage targeting molecule or moiety (e.g., a cartilage targeting peptide, such as WYRGRL). In any aspect or embodiment described herein, the assembled cartilage-targeting JBNP increases the retention time in articular cartilage up to 14 days. In any aspect or embodiment described herein, the targeting ability, retention time, and clearance of a JBNP in the knee joint can be examined by an intra-articular injection of a JBNP comprising an mRNA reported (e.g., a fluorescent marker), optionally including the targeting molecule/moiety/peptide WYRGRL (SEQ ID NO: 47), and fluorescence detected via IVIS® imaging.

In any aspect or embodiment described herein, the JBNP comprises, or has attached thereto, the cartilage targeting molecule or moiety WYRGRL (SEQ ID NO:47), and optionally an mRNA reporter (e.g., Fluc mRNA), thereby delivering the agent or therapeutic agent (e.g. mRNA) to cartilage, such as to joints. In any aspect or embodiment described herein, the targeting efficiency of the cartilage targeting-JBNP (e.g. WYRGRL-JBNP) with a particular agent or therapeutic agent can be examined by an intra-articular injection of the cartilage targeting-JBNP comprising a mRNA for a fluorescent marker (e.g., Fluc mRNA), and fluorescence signal examined with IVIS® imaging, which can also be used to confirm and quantify mRNA translation. For example, in any aspect or embodiment described herein, histological section can be taken and stained with 4′,6-diamidino-2-phenylindole (DAPI) examine delivery via fluorescence microscopy.

The JBNP was shown to not cause significant toxicity in the knee joint. Histological assessment was performed, such as hematoxylin and cosin (H&E) staining, Safranin O staining, and alcian blue staining.

In any aspect or embodiment described herein, the pH of pharmaceutical composition including the self-assembled nanomaterial may be a physiological pH.

This disclosure is further illustrated by the following non-limiting examples.

### Specific Embodiments

In any aspect or embodiment described herein, the JBNP comprises a targeting molecule comprising the amino acid sequence WYRGRL and miR 140 (WYRGRL-JBNP-miR140). In any aspect or embodiment described herein, the WYRGRL-JBNP-miR140 is used and effective in the treatment, prevent, or amelioration of one or more symptoms of osteoarthritis.

In any aspect or embodiment described herein, the JBNP comprises CRISPR targeting sgIL-1R (JBNP-CRISPR-sgIL-1R). In any aspect or embodiment described herein, the JBNP-CRISPR-sgIL-1R is used in the treatment, prevent, or amelioration of one or more symptoms of osteoarthritis, such as inflammation (e.g., inflammation in the knee joint).

In any aspect or embodiment described herein, the JBNP comprises IL-1R siRNA (JBNP-IL-1R siRNA). In any aspect or embodiment described herein, the JBNP-IL-1R siRNA is used and effective in the treatment, prevent, or amelioration of one or more symptoms of osteoarthritis, such as inflammation (e.g., inflammation in the knee joint).

In any aspect or embodiment described herein, the JBNP comprises IL-1RA mRNA (JBNP-IL-1RA mRNA). In any aspect or embodiment described herein, the JBNP-IL-1RA mRNA is used and effective in the treatment, prevent, or amelioration of one or more symptoms of osteoarthritis, such as inflammation (e.g., inflammation in the knee joint). In any aspect or embodiment described herein, the mRNA sequence of an IL-1RA is:

(SEQ ID NO: 89)
ATGGAAATCTGCAGAGGCCTCCGCAGTCACCTAATCACTCTCCTCCTCT
TCCTGTTCCATTCAGAGACGATCTGCCGACCCTCTGGGAGAAAATCCAG
CAAGATGCAAGCCTTCAGAATCTGGGATGTTAACCAGAAGACCTTCTAT
CTGAGGAACAACCAACTAGTTGCTGGATACTTGCAAGGACCAAATGTCA
ATTTAGAAGAAAAGATAGATGTGGTACCCATTGAGCCTCATGCTCTGTT
CTTGGGAATCCATGGAGGGAAGATGTGCCTGTCCTGTGTCAAGTCTGGT
GATGAGACCAGACTCCAGCTGGAGGCAGTTAACATCACTGACCTGAGCG
AGAACAGAAAGCAGGACAAGCGCTTCGCCTTCATCCGCTCAGACAGTGG
CCCCACCACCAGTTTTGAGTCTGCCGCCTGCCCCGGTTGGTTCCTCTGC
ACAGCGATGGAAGCTGACCAGCCCGTCAGCCTCACCAATATGCCTGACG
AAGGCGTCATGGTCACCAAATTCTACTTCCAGGAGGACGAGTAG.

In any aspect or embodiment described herein, the JBNP comprises IL-1RA peptide mRNA (JBNP-IL-1RA peptide mRNA). In any aspect or embodiment described herein, the JBNP-IL-1RA peptide mRNA is used and effective in the treatment, prevent, or amelioration of one or more symptoms of osteoarthritis, such as inflammation (e.g., inflammation in the knee joint). In any aspect or embodiment described herein, the mRNA sequence of an IL-1RA peptide is:

(SEQ ID NO: 90)
ATGCGGCCTAGCGGCAGAAAGTCTAGCAAGATGCAGGCCTTTAGAATCT
GGGACGTTAATCAGAAAACCTTCTACCTCAGAAACAACCAGCTGGTCGC
CGGCTACCTGCAGGGCCCCAACGTGAATCTGGAAGAGAAGATCGACGTG
GTGCCAATCGAGCCTCACGCCCTGTTTCTGGGCATCCACGGCGGAAAAA
TGTGCCTGTCTTGTGTGAAGTCCGGAGATGAGACAAGACTGCAACTGGA
AGCCGTGAACATTACAGACCTGAGCGAGAACCGGAAGCAGGACAAGCGG
TTCGCCTTCATCAGAAGCGACAGCGGCCCTACAACCAGCTTCGAGAGCG
CCGCTTGCCCCGGCTGGTTCCTGTGCACCGCCATGGAAGCTGATCAGCC
TGTGTCCCTGACCAACATGCCTGATGAAGGCGTGATGGTGACCAAGTTC
TACTTCCAGGAGGACGAGATGTGA.

In any aspect or embodiment described herein, the JNBT or JNBP includes a lysine or arginine side chain. In any aspect or embodiment described herein, the JNBT or JNBP includes a lysine or arginine side chain to target the self-assembled nanomaterials to the liver.

In any aspect or embodiment described herein, the JNBT or JNBP includes a histine side chain. In any aspect or embodiment described herein, the JNBT or JNBP includes a histidine side chain to target the self-assembled nanomaterials to organs other than the liver.

In any aspect or embodiment described herein, the JBNT or JBNP comprises a cartilage targeting molecule, such as WYRGRL or RLDPTSYLRTFW. In any aspect or embodiment described herein, the JBNT or JBNP comprises a cartilage targeting molecule, such as WYRGRL or RLDPTSYLRTFW, is used in a method to deliver the JBNT or JBNP to cartilage.

In any aspect or embodiment described herein, the JBNP comprises an agent that is mRNA, optionally with a targeting molecule, and optionally a reporter or label (e.g., tdTomato mRNA, Cy5, AF488, etc.). In any aspect or embodiment described herein, the JBNP comprises an agent that is mRNA (JBNP-mRNA), optionally with a targeting molecule, and optionally a reporter/label (e.g., tdTomato mRNA, Cy5, AF488, etc) that is delivered to the liver, kidney, brain, lung, spleen, lymph nodes, bone, muslbe, heart, pancreas, intestine, solid tumor, or a combination thereof.

In any aspect or embodiment described herein, the JBNP comprises Cas9mRNA and sgRB1. In any aspect or embodiment described herein, the JBNP comprising Cas9mRNA and sgRB1 is used in a method to deliver the sgRB1 to (and edit) liver cells.

In any aspect or embodiment described herein, the JBNP comprises CRISPR (e.g., Cas9mRNA and gRNA) and a tumor targeting moiety. In any aspect or embodiment described herein, the JBNP comprising CRISPR (e.g., Cas9mRNA and gRNA) and a tumor targeting moiety is used in a method of delivering and editing the DNA of a tumor cell.

In any aspect or embodiment described herein, the JBNT and JBNP due not cause acute toxicity, an innate immune response, an adaptive immune response, or a combination thereof, when administered.

In any aspect or embodiment described herein, the side chain of the JBNT is modified to facilitate a particular type of cellular update. For example, in any aspect or embodiment described herein, lysine-based JBNTs are used to facilitate cellular uptake of JBNPs through micropinocytosis. By way of further example, in any aspect or embodiment described herein, arginine-based JBNTs are used to facilitate cellular update of JBNPs through clathrin-mediated endocytosis.

In any aspect or embodiment described herein, the JBNP comprises lysine-based JBNTs, which have significantly better endosomal escape that appears to be due to the proton sponge effect

In any aspect or embodiment described herein, the JBNP comprises arginine-based JBNTs, which have significantly enhanced endosomal escape that appears to be due to pore-forming effect to escape early endosome.

In any aspect or embodiment described herein, the JBNP of the present disclosure have lower cell-cytotoxicity than lipid nanoparticles, polymer nanoparticles (such as poly-1-lysine (PLL) nanoparticles and polyethylenimine (PEI) nanoparticles), single-wall nanotubes, or a combination thereof.

In any aspect or embodiment described herein, the JBNP comprises Cas9mRNA-eGFP and gRNA-ATTO550.

### Examples

Materials: All reagents and solvents disclosed herein were obtained from commercial suppliers and used without further purification. Commercial suppliers include Sigma-Aldrich, Alfa Aesar, Fisher Scientific and Thermo Fisher.

### Example 1

Reaction scheme 1 illustrates the formation of compound S4.

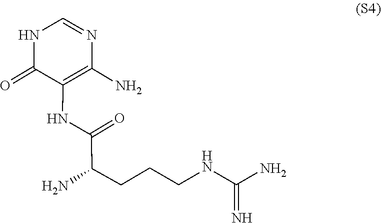 [SMILES: N=C(N)NCCC[C@H](N)C(=O)Nc1c(N)nc[nH]c1=O]

Synthesis of compound S3. To a solution of commercially available compounds S1 (63 mg) and S2 (271 mg) in dimethylformamide (DMF, 2 mL), hydroxybenzotriazole (HOBt, 135 mg), 1-ethyl-3-(3-dimethylaminopropyl) carbodiimide (EDCI, 192 mg), and N,N-Diisopropylethylamine (DIPEA, 173 μL) were added, and the resulting solution was stirred overnight (˜ 8 hours) at room temperature. The reaction mixture was quenched with aqueous NH4Cl and extracted with ethyl acetate (EA). The combined organic layers were dried with MgSO₄ and concentrated under vacuum. The crude product was purified by flash column chromatography (3% MeOH/DCM) to produce compound S3 (199 mg, 31%) as a white solid. 1H NMR (500 MHZ, Chloroform-d) δ 9.63 (brs, 1H), 7.81 (s, 1H), 7.43-7.27 (m, 10H), 5.99 (brs, 1H), 5.61 (brs, 1H), 5.32-5.21 (m, 2H), 5.19-5.07 (m, 2H), 4.34 (s, 1H), 4.16-3.92 (m, 2H), 2.00-1.70 (m, 4H), 1.41 (s, 9H). HRMS (ESI) [M+H]⁺ calculated for 651.2885, found 651.2809.

Synthesis of compound $4. Compound S3 (199 mg) was added into a 94% trifluoroacetic acid (TFA)/thioanisole (4.9 mL) solution. After stirring at room temperature for 72 hours, diethyl ether (Et20) was added. A white precipitate formed and then was centrifuged down. After decanting the supernatant, the white precipitate was washed with Et2O to yield a crude product. The crude product was purified using HPLC to produce compound S4 (111 mg, 94%). HRMS (ESI) [M+H]⁺ calculated for 283.1625, found 283.1654.

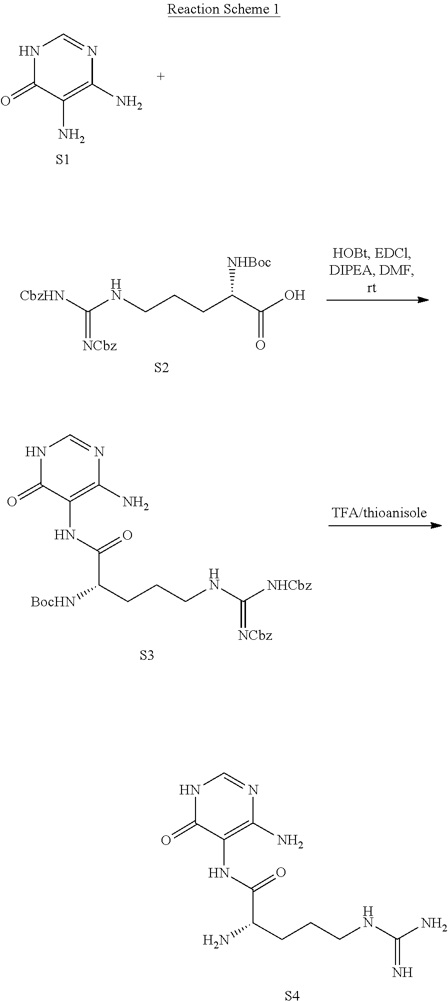

### Example 2

Reaction scheme 2 illustrates the formation of compound S10.

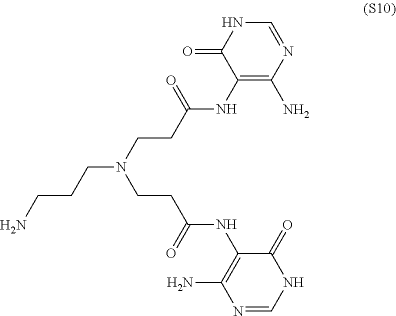 [SMILES: NCCCN(CCC(=O)Nc1c(N)nc[nH]c1=O)CCC(=O)Nc1c(N)nc[nH]c1=O]

Synthesis of compound S7. To the solution of commercially available S5 (211 mg) and S6 (0.27 mL) in MeOH (5 mL) was added Et3N (0.21 mL), the resulting solution was stirred overnight (˜ 8 hours) at room temperature. The volatiles were removed under vacuum and the resulting material was purified by flash column chromatography (100% EA) to produce compound S7 (190 mg, 55%) as a colorless oil. 1H NMR (500 MHZ, Chloroform-d) δ 5.11 (s, 1H), 3.67 (s, 6H), 3.12 (s, 2H), 2.73 (brm, 4H), 2.44 (brm, 6H), 1.61 (s, 2H), 1.56 (s, 2H), 1.43 (s, 9H). HRMS (ESI) [M+H]⁺ calculated for 347.2177, found 347.2170.

Synthesis of compound S9. To the solution of compound S7 (168 mg) in THF (3 mL) was added aq. NaOH (1 M, 2 mL), the resulting solution was stirred for 2 hours at room temperature. After completion, the reaction mixture was adjusted to pH 6 using concentrated HCl. To the acidified reaction mixture were added compounds S1 (62 mg), HOBt (66 mg) and EDCI (94 mg), the resulting mixture was stirred for 20 hours at room temperature. The reaction mixture was quenched with aq. NaHCO₃ and extracted with ethyl acetate (EA). The combined organic layers were dried with MgSO₄ and concentrated under vacuum. The crude product was used directly in the next step without further purification.

Synthesis of compound S10. Crude compound S9 (2 mg) was dissolved in TFA (0.2 mL) and the resulting solution was stirred for 15 min. Diethyl ether (Et20) was added and a brown precipitate formed and was then centrifuged down. After pouring supernatant out, the brown precipitate was then washed with Et20 to yield crude product. The crude product was purified using HPLC to produce compound S10 (1 mg). HRMS (ESI) [M+H]⁺ calculated for 435.2211, found 435.2309.

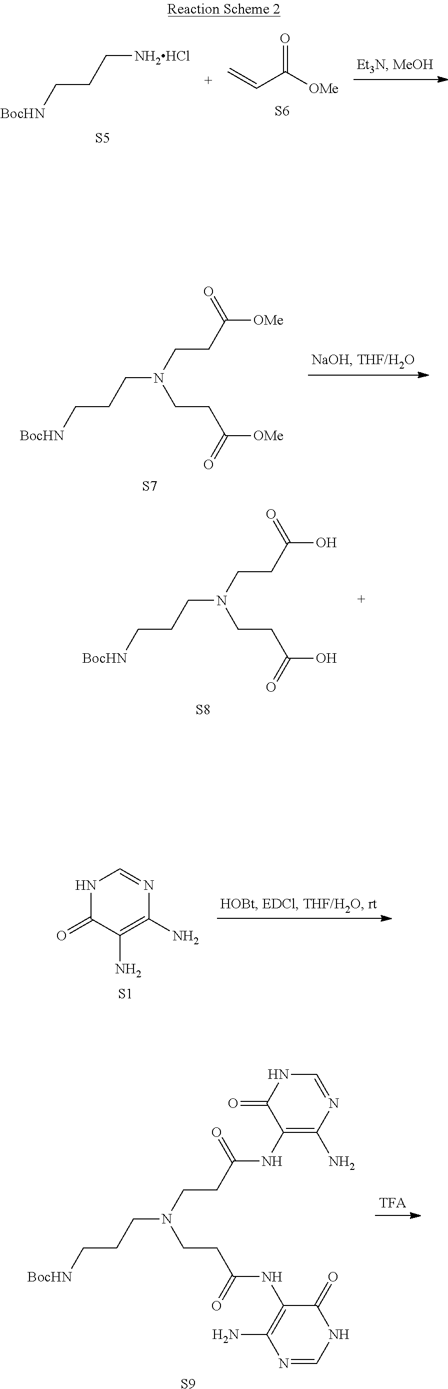

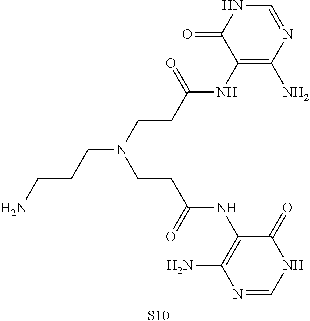 [SMILES: NCCCN(CCC(=O)Nc1c(N)nc[nH]c1=O)CCC(=O)Nc1c(N)nc[nH]c1=O ; "S10"]

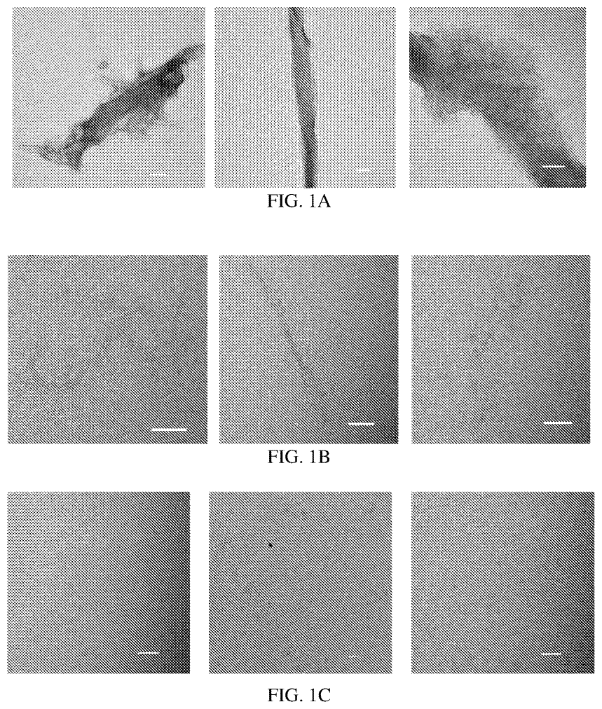

TEM was performed to characterize the morphology of the JBNTs prepared as described in Examples 1 and 2. FIG. 1A shows the structure of JBNTs composed of compound S4 (single ring nanotubes), and FIG. 1B shows the structure of JBNTs composed of compound S10 (twin single ring nanotubes).

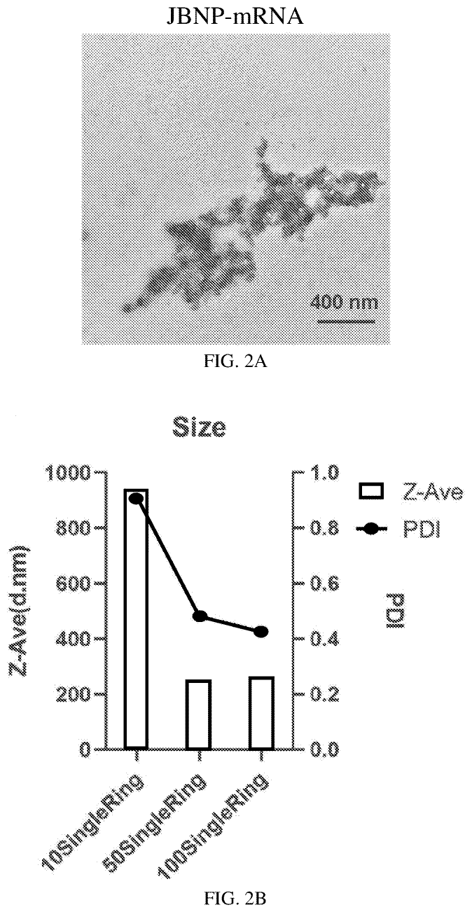

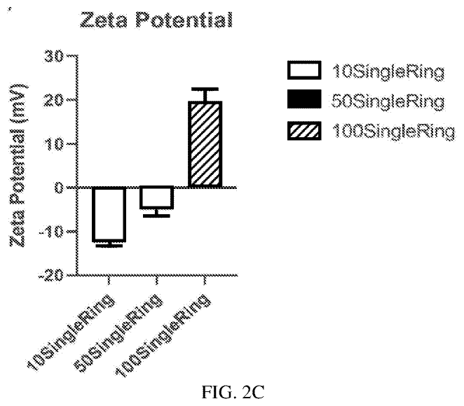

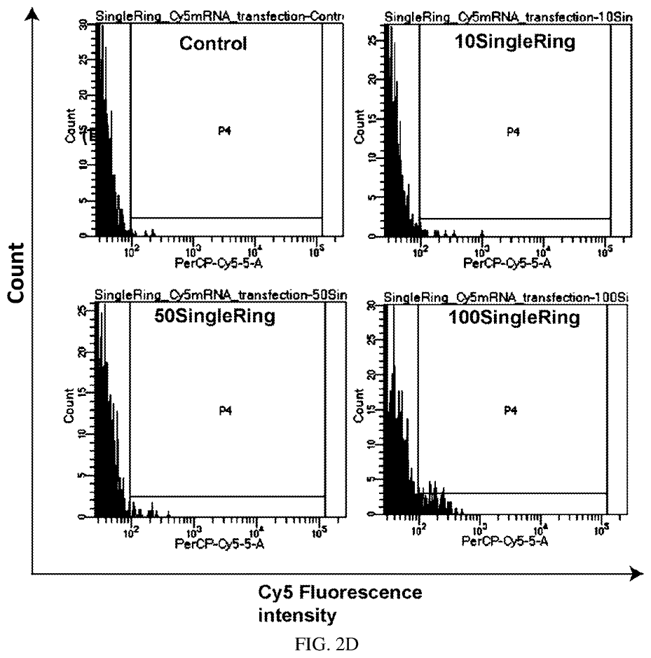

FIGS. 2A, 2B, 2C, and 2D are TEM images of Janus Base Nanopiece encapsulating mRNA (JBNP-mRNA) and demonstrate cellular delivery. FIG. 2A shows the transmission electron microscope (TEM) image of JBNP-mRNA. FIG. 2B shows the hydrodynamic size of the JBNP-mRNAs. FIG. 2C shows the surface charge of JBNP-mRNAs. FIG. 2D shows the delivery of mRNAs to the human chondrocytes (C28/12).

Example 3. Examining the ability of miR 140 delivery with WYRGRL-JBNP to slows down or present osteoarthritis (OA) progression. OA severity is assessed by MANKIN score prior to administering treatment (saline, JBNP-miR140, JBNP-scrambled miRNA, or JBNP-without miR140) via an intra-articular injection into surgical destabilization of the medial meniscus (DMM) model mice 1-month post-surgery. The following behavioral studies are performed to examine treatment efficacy: Y-Maze Spontaneous Alteration, Open Field, Object Recognition Test, and Fear Conditioning. DAPI staining, H&E staining, and Safranin-O staining of histological sections is used to demonstrate effective targeting and delivery via fluorescent microscopy.

Example 4. Examining the ability of JBNP-CRISPR targeting sgIL-1R to treat OA, slowing down the progression of OA, and inhibit inflammation. OA severity is assessed by MANKIN score prior to administering treatment (saline, JBNP-CRISPR-sgIL-1R, JBNP-CRISPR-sgNeg, or JBNP-CRISPR-without sgRNA) via an intra-articular injection into surgical destabilization of the medial meniscus (DMM) model mice 2 months post-surgery or 129SVE-M wild-type mice. DAPI staining, H&E staining, and Safranin-O staining of histological sections examines OA progress via fluorescent microscopy. The following behavioral studies are performed to examine treatment efficacy: Y-Maze Spontaneous Alteration, Open Field, Object Recognition Test, and Fear Conditioning.

Example 5. Examining the ability of JBNP-IL-1R siRNA to inhibit OA-related inflammation in the knee joint. OA severity is assessed by MANKIN score prior to administering treatment (saline, JBNP-IL-1R siRNA, JBNP-negative siRNA, JBNP-without siRNA) via an intra-articular injection into surgical destabilization of the medial meniscus (DMM) model (3-month-old male) mice 1-month post-surgery. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning.

Example 6. Examining the ability of JBNP-IL-1RA mRNA to inhibit OA-related inflammation in the knee joint. OA severity is assessed by MANKIN score prior to administering treatment (saline, JBNP-IL-1RA mRNA, JBNP-scrambled mRNA, JBNP-without mRNA) via an intra-articular injection into surgical destabilization of the medial meniscus (DMM) model (3-month-old male) mice 1-month post-surgery. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an, mRNA sequence for IL-1RA is:

(SEQ ID NO: 89)
ATGGAAATCTGCAGAGGCCTCCGCAGTCACCTAATCACTCTCCTCCTCT
TCCTGTTCCATTCAGAGACGATCTGCCGACCCTCTGGGAGAAAATCCAG
CAAGATGCAAGCCTTCAGAATCTGGGATGTTAACCAGAAGACCTTCTAT
CTGAGGAACAACCAACTAGTTGCTGGATACTTGCAAGGACCAAATGTCA
ATTTAGAAGAAAAGATAGATGTGGTACCCATTGAGCCTCATGCTCTGTT
CTTGGGAATCCATGGAGGGAAGATGTGCCTGTCCTGTGTCAAGTCTGGT
GATGAGACCAGACTCCAGCTGGAGGCAGTTAACATCACTGACCTGAGCG
AGAACAGAAAGCAGGACAAGCGCTTCGCCTTCATCCGCTCAGACAGTGG
CCCCACCACCAGTTTTGAGTCTGCCGCCTGCCCCGGTTGGTTCCTCTGC
ACAGCGATGGAAGCTGACCAGCCCGTCAGCCTCACCAATATGCCTGACG
AAGGCGTCATGGTCACCAAATTCTACTTCCAGGAGGACGAGTAG.

Example 7. Examining the ability of JBNP-IL-1RA peptide mRNA to inhibit OA-related inflammation in the knee joint. OA severity is assessed by MANKIN score prior to administrating treatment (saline, JBNP-IL-1RA peptide mRNA, JBNP-scrambled mRNA, JBNP-without mRNA) via an intra-articular injection into surgical destabilization of the medial meniscus (DMM) model (3-month-old) mice 1-month post-surgery. The IL-1RA peptide is an interleukin-1 receptor antagonist peptide. Safranin-O staining is performed on histological sections and examined via fluorescent microscopy. The following behavioral studies are performed to examine treatment efficacy: Y-Maze Spontaneous Alteration, Open Field, Object Recognition Test, and Fear Conditioning. An exemplary, nonlimiting example of an, mRNA sequence for an IL-1RA peptide is:

(SEQ ID NO: 90)
ATGCGGCCTAGCGGCAGAAAGTCTAGCAAGATGCAGGCCTTTAGAATCT
GGGACGTTAATCAGAAAACCTTCTACCTCAGAAACAACCAGCTGGTCGC
CGGCTACCTGCAGGGCCCCAACGTGAATCTGGAAGAGAAGATCGACGTG
GTGCCAATCGAGCCTCACGCCCTGTTTCTGGGCATCCACGGCGGAAAAA
TGTGCCTGTCTTGTGTGAAGTCCGGAGATGAGACAAGACTGCAACTGGA
AGCCGTGAACATTACAGACCTGAGCGAGAACCGGAAGCAGGACAAGCGG
TTCGCCTTCATCAGAAGCGACAGCGGCCCTACAACCAGCTTCGAGAGCG
CCGCTTGCCCCGGCTGGTTCCTGTGCACCGCCATGGAAGCTGATCAGCC
TGTGTCCCTGACCAACATGCCTGATGAAGGCGTGATGGTGACCAAGTTC
TACTTCCAGGAGGACGAGATGTGA.

Example 8. Amino acid side change modification can be utilized to target self-assembled nanomaterials of the present disclosure to organs of interest. The protein corona of the self-assembled nanomaterials described herein and compositions comprising the same can be further modify through the modification of the side chain of the amino acid residue. Assembled JBNPs are incubated with mice serum for 30 minutes. Then, centrifuge for 15 minutes at 13800 RCF at 4° C. The supernatant is discarded and pellet washed three times with phosphate-buffered saline. The proteins are separates via SDS-PAGE and the protein bands identified via Liquid Chromatography-tandem Mass spectrometry (LC/MS/MS). Lysine and arginine side chains bind Apolipoprotein E (APOE) family and believed to target the self-assembled nanomaterials of the present disclosure to the liver. By way of further example, it is believed that the histidine side chain in the protein corona composition targets the self-assembled nanomaterials of the present disclosure will target to organs other than liver.

Example 9. Examining the ability of the JBNP of the present disclosure to actively target cartilage and enhance half-life when a cartilage targeting peptide (WYRGRL) is attached to the JBNT. The cartilage targeting peptide WYRGRL (SEQ ID NO:47) is attached to JBNP with an mRNA reporter and the assembled WYRGRL-JBNP with the mRNA-dye is transferred to human chondrocytes C28/12 cells. FACS is used to quantify the targeting ability of the WYRGRL-JBNP to target cartilage.

Example 10. Examining the ability of the JBNP of the present disclosure to actively target cartilage and enhance half-life when a cartilage targeting peptide (RLDPTSYLRTFW) is attached to the JBNT. The cartilage targeting peptide RLDPTSYLRTFW (SEQ ID NO:46) is attached to JBNP with an mRNA reporter and the assembled RLDPTSYLRTFW-JBNP with the mRNA-dye, and is transferred to human chondrocytes C28/12 cells. FACS is used to quantify the targeting ability of the RLDPTSYLRTFW-JBNP to target cartilage.

Example 11. Examining the ability of the JBNP of the present disclosure to deliver an agent to the liver. JBNP-mRNA Cy5 is intravenously administered to BALB/cJ mice. Biodistribution of the JBNP-mRNA Cy5 is monitored via IVIS® imaging. Then, the transfected liver is homogenized. Western blot and real-time reverse transcriptase quantitative polymerase chain reaction (RT-qPCR) are used to determine the transfection efficiency.

Example 12. Examining the ability of the JBNP of the present disclosure to deliver an agent to the kidney. JBNP-mRNA Cy5 is intravenously administered to BALB/cJ mice. Biodistribution of the JBNP-mRNA Cy5 is monitored via IVIS® imaging. Then, the transfected kidney is homogenized. Western blot and RT-qPCR was used to determine the transfection efficiency.

Example 13. Examining the ability of the JBNP of the present disclosure to deliver an agent to the brain. JBNP-mRNA Cy5 is intravenously administered to BALB/cJ mice. Biodistribution of the JBNP-mRNA Cy5 is monitored via IVIS® imaging. Then, the transfected brain is homogenized. Western blot and RT-qPCR are used to determine the transfection efficiency.

Example 14. Examining the ability of the JBNP of the present disclosure to deliver an agent to the lung. JBNP-mRNA Cy5 is intravenously administered to BALB/cJ micc. Biodistribution of the JBNP-mRNA Cy5 is monitored via IVIS® imaging. Then, the transfected lung is homogenized. Western blot and RT-qPCR are used to determine the transfection efficiency.

Example 15. Examining the ability of the JBNP of the present disclosure to deliver an agent to the spleen and lymph nodes. JBNP-mRNA Cy5 is intravenously administered to BALB/cJ mice. Biodistribution of the JBNP-mRNA Cy5 is monitored via IVIS® imaging. Then, the transfected spleen and lymph nodes are homogenized. Western blot and RT-qPCR are used to determine the transfection efficiency.

Example 16. Examining the ability of the JBNP of the present disclosure to deliver an agent to bone. JBNP-mRNA Cy5 is intravenously administered to BALB/cJ mice. Biodistribution of the JBNP-mRNA Cy5 is monitored via IVIS® imaging. Then, the transfected bone is homogenized. Western blot and RT-qPCR are used to determine the transfection efficiency.

Example 17. Examining the ability of the JBNP of the resent disclosure to deliver an agent to muscle. JBNP-mRNA Cy5 is intravenously administered to BALB/cJ mice. Biodistribution of the JBNP-mRNA Cy5 is monitored via IVIS® imaging. Then, the transfected muscle is homogenized. Western blot and RT-qPCR are used to determine the transfection efficiency.

Example 18. Examining the ability of the JBNP of the present disclosure to deliver an agent to the heart. JBNP-mRNA Cy5 is intravenously administered to BALB/cJ mice. Biodistribution of the JBNP-mRNA Cy5 is monitored via IVIS® imaging. Then, the transfected heart is homogenized. Western blot and RT-qPCR are used to determine the transfection efficiency.

Example 19. Examining the ability of the JBNP of the present disclosure to deliver an agent to the pancreas. JBNP-mRNA Cy5 is intravenously administered to BALB/cJ mice. Biodistribution of the JBNP-mRNA Cy5 is monitored via IVIS® imaging. Then, the transfected pancreas is homogenized. Western blot and RT-qPCR are used to determine the transfection efficiency.

Example 20. Examining the ability of the JBNP of the present disclosure to deliver an agent to the intestine. JBNP-mRNA Cy5 is intravenously administered to BALB/cJ mice. Biodistribution of the JBNP-mRNA Cy5 is monitored via IVIS® imaging. Then, the transfected heart is homogenized. Western blot and RT-qPCR are used to determine the transfection efficiency.

Example 21. Examining the ability of the JBNP of the present disclosure to deliver an agent to a solid tumor. JBNP-mRNA Cy5 is intravenously administered to xenograft model. Biodistribution of the JBNP-mRNA Cy5 is monitored via IVIS® imaging. Then, the transfected solid tumor is homogenized. Western blot and RT-qPCR are used to determine the transfection efficiency. The mouse/mice are monitored for tumor size, weight, complete blood count (CBC), tumor necrosis factor-alpha (TNF-alpha) levels, interferon-gamma (IFN-gamma) levels, immunoglobulin G (IgG) levels, immunoglobulin M (IgM) levels, and toxicity.

Example 22. Optimizing dosage and delivery of JBNP-CRISPR. Optimized dosage and optimized delivery time for JBNP-CRISPR is determined by performing gene editing with the Ai14 mouse model. JBNP-CRISPR that is composed of Cas9mRNA (0.25 mg/kg) and sgLOXp is intravenously injected via tail vein injection or retro-orbital injection for 2-7 days. Positive tdTomato signals is monitored/detected via IVIS® imaging. Next-Generation Sequencing (NGS) is used to quantify gene editing efficiency in the target organ.

Example 23. Examining the ability of JBNP-CRISPR to target and edit the DNA of major organs. Assembled JBNP-CRISPR composed of Cas9mRNA and sgRB1 is intravenously injected to BALB/cJ mice for 7-28 days. Sanger sequencing or Next Generation Sequencing (NGS) is used to explore the RBI gene editing efficiency.

Example 24. Examining the ability of JBNP-CRISPR to target and edit the DNA of different organs via the attachment of a targeting molecule or moiety. JBNP-CRISPR with a kidney targeting molecule or moiety can target the kidney and edit the DNA of kidney cells. JBNP-CRISPR with a heart targeting molecule or moiety can target the heart and edit the DNA of heart cells. JBNP-CRISPR with a spleen targeting molecule or moiety can target the spleen and edit the DNA of spleen cells. JBNP-CRISPR with lymph nodes targeting molecule or moiety can target the lymph nodes and edit the DNA of lymph node cells. JBNP-CRISPR with a lung targeting molecule or moiety can target the lungs and edit the DNA of lung cells. JBNP-CRISPR with a muscle targeting molecule or moiety can target muscles and edit the DNA of muscle cells. JBNP-CRISPR with a pancreas targeting molecule or moiety can target the pancreas and edit the DNA of pancreatic cells. JBNP-CRISPR with an intestine targeting molecule or moiety can target the intestines and edit the DNA of intestine cells.

Example 25. Examining the ability of JBNP-CRISPR to target and edit the DNA of solid-tumors via the attachment of a targeting molecule or moiety. JBNP-CRISPR with a tumor targeting molecule or moiety can target a solid-tumor and edit the DNA of tumor cells.

Example 26. Examining whether the JBNP of the present disclosure causes any acute toxicity. Acute toxicity is examined by tail-vein injection of the JBNP dosage (dosage optimized for each treatment) every 5 days for 20 days. H&E staining of histological sections of the liver, spleen, kidney, heart, pancreas, lung, brain, bone, and muscle is performed.

Examples 27. Examining whether the JBNP of the present disclosure causes an innate immune response when administered. JBNP is administered intravenously to BALB/cJ mice every 3 days. Serum (30 μl) is collected from submandibular laceration for each injection time point. IFN-gamma, IL-1beta, IL-6, IL-10, IL-12 (p70), TNF-alpha, MCP-1, MIP-2, MIG, IL-2, IL-5, and IL-17 are examined via Immunology Multiplex Assay and ELISA.

Examples 28. Examining whether the JBNP of the present disclosure causes an adaptive immune response when administered. JBNP is administered intravenously to BALB/cJ mice every 3 days, for 9 days. Serum is collected prior to receiving JBNP and after 10 day. Then, IgM and IgG levels are examined via ELISA. Serum IgM and IgG antibody levels on day 10 are compared to the pre-injection baseline.

Example 29. Examining cellular uptake mechanism of the JBNP based upon the modification or composition of the JBNT. Cells are pretreated with several endocytic inhibitors, including macropinocytosis inhibitors (Latrunculin A, cytochalasin D), clathrin-mediated inhibitor (chlorpromazine), and a caveolae-mediated inhibitor (methyl-b-cyclodextrin). Then, the assembled JBNP-siRNA AF488 is transfected into cells. After a 24-hour incubation, FACS is used to quantify the AF488 signal and to determine the uptake mechanism. Different side-chains likely have different cellular uptake mechanisms. For example, cellular uptake of JBNP comprising lysine-based JBNTs may be through macropinocytosis. Furthermore, cellular update of JBNP comprising arginine-based JBNT may be through clathrin-mediated endocytosis.

Example 30. Examining endosomal escape mechanism of JBNPs based upon the modification or composition of the JBNT. Endosomal escape is examined for JBNPs comprising different types of JBNT of the present disclosure. The endosomes are examined by staining the cells contacted with the tested JBNP with LysoTracker™ RED which stains for and visualized endosomes in cells. The degree of colocalization is quantified based on Pearson's correlation coefficient (r) using the Image J software following the colocalization threshold and coloc2 plugin. Bafilomycin Al and chloroquine inhibitors are used in a pre-treatment to demonstrate the proton-sponge effect. Calcein assay and modeling are used to explore the pore-formation to escape the endosomes. JBNP comprising lysine-based JBNTs likely have significantly better endosomal escape due to the proton sponge effect. Furthermore, JBNP comprising arginine-based JBNTs likely have significantly enhanced endosomal escape due to pore-forming effect to escape early endosome.

Example 31. Examining whether the JBNP of the present disclosure are highly biocompatible with low cell-toxicity. Human chondrocytes are seeded (5000 cells/well) and incubated overnight. Then, cell viability of various vectors, including JBNP, are determined by Cell Counting Kit-8 assay (Sigma). After coincubation for 24 hours, the absorbance is obtained by a microplate reader.

Example 32. C28/12 cells are seeded in a 24-well plate (5000 cells/well) and incubated overnight. Co-assembled JBNP-Cas9mRNA-cGFP and gRNA-ATTO550 dye are directly transferred to the well and transfected for 72 hours. Then, FACS is performed to quantify the co-delivery in a cell.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A self-assembled nanomaterial comprising a Janus base nanotube and a biologically active molecule covalently or non-covalently adhered thereto, wherein the Janus base nanotube comprises at least one compound represented by Formulas I, II, III, IV, V, VI, VII, VIII, IX, or a pharmaceutically acceptable salt thereof: 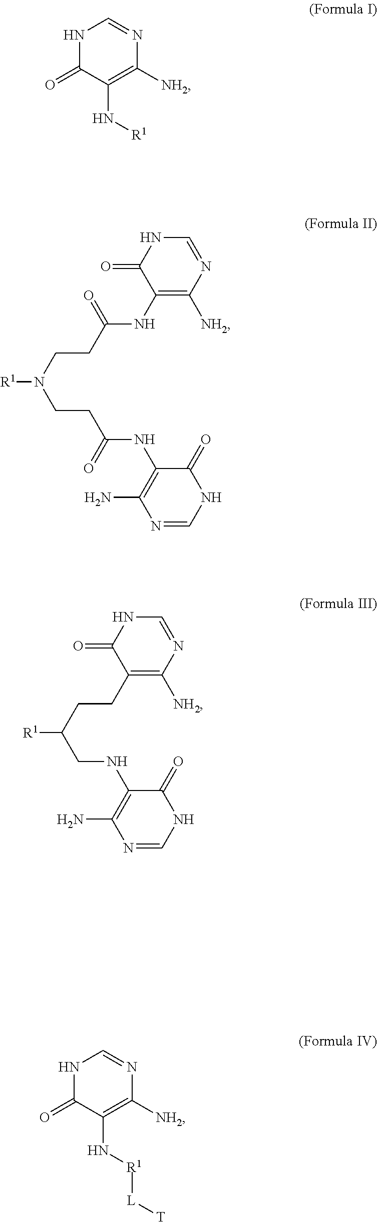 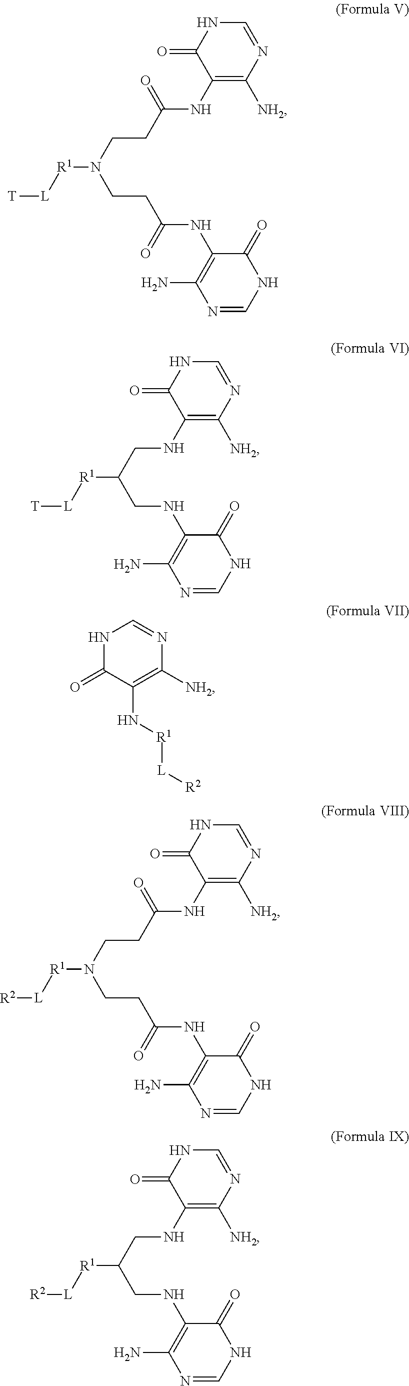
wherein,
R¹ of Formulas I-III is H, —(CH₂)ⱼH, —(CH₂CH₂O)ₖH, —(CH₂CH₂NH)ₘH, an α-amino acid, β-amino acid, an α-polypeptide, or a β-polypeptide;
R¹ of Formulas IV-LX is absent, —(CH₂)ⱼ, —(CH₂CH₂O)ₖ—, —(CH₂CH₂NH)ₘ—, an α-amino acid, a β-amino acid, an α-polypeptide, or a β-polypeptide;
each j, k, and m is independently 1-200;
L is a bond or a linker group;
T is H, a biologically active molecule (e.g., an agent, such as a therapeutic agent), or a targeting molecule; and
R³ is H or a coating/protecting material.

2. The self-assembled nanomaterial of claim 1, wherein L is the linker group and is selected from an acid-cleavable group, a reducible disulfide group, an α-amino acid, a β-amino acid, an α-polypeptide, a β-polypeptide, a trans-cyclooctene group, a thioether-containing group, an enzyme cleavable group, a stimuli-responsive group, or a combination thereof.

3. The self-assembled nanomaterial of claim 2, wherein
the acid-cleavable linker is selected from N-acyl hydrazone, a carbonate group, or an ester group;
the reducible disulfide linker is selected from N-succinimidyl-4-(2-pyridyldithio) pentanoate (SPP), N-succinimidyl-4-(2-pyridyldithio) butyrate (SPDB), or 4-(+′-acetylphenoxy) butanoic acid (AcBut), Val-Cit dipeptide, Phe-Lys dipeptide, an α-methyl substituted disulfide, an engineered cysteine residue, or a thiol-containing maytansinoid;
the stimuli-responsive linker is selected from a trans-cyclooctene linker, or a thioether-containing linker; or
the enzyme cleavable linker is selected from GPLGOAGQ (SEQ ID NO:91), GDEVEAPKGC (SEQ ID NO: 92), citrulline-valine, a glycosidase-cleavable linker, a β-glucoronidase-cleavable linker, a β-Galactosidase-cleavable linker, a phosphatase-cleavable linker, a pyrophosphate-containing linker, a dipeptide-containing linker, Phe-Lys-PABC (para-aminobenzyl carbamate), a Val-Cit-PABC containing linker, a Glu-Val-Cit-containing linker, or a Val-Ala containing linker.

4. The self-assembled nanomaterial of claim 1, wherein R² is the coating/protecting material and is selected from a polymer, a peptide, a polypeptide, a lipid-based material, phosphate ester, a biomimetic membrane, or a combination thereof.

5. The self-assembled nanomaterial of claim 1, wherein R² is the coating/protecting material and is selected from polyethylene glycol, chitosan, hyaluronic acid, a poloxamer, polyvinyl alcohol, a polysaccharide, a neutral or negatively charged poly(amino acid), phytochelatin, a self-peptide, an antithrombotic peptide, or a combination thereof.

6. The self-assembled nanomaterial of claim 1, wherein T is the targeting molecule and is selected from a small molecule, a polymer (such as an amphiphilic polymer), an aptamer, a peptide, a protein, a polysaccharide, a polyunsaturated fatty acid, a carbohydrate, or a combination thereof.

7. The self-assembled nanomaterial of claim 1, wherein T is the biologically active molecule and the biologically active molecule is covalently adhered to the self-assembled nanomaterial.

8. The self-assembled nanomaterial of claim 1, wherein I is the biologically active molecule and the biologically active molecule is noncovalently adhered to the self-assembled nanomaterial.

9. The self-assembled nanomaterial of claim 8, wherein the biologically active molecule is at least partially encapsulated by the self-assembled nanomaterial.

10. The self-assembled nanomaterial of claim 1, wherein the biologically active molecule comprises a nucleic acid, a protein, a peptide, a small molecule drug, or a combination thereof.

11. The self-assembled nanomaterial of claim 1, wherein the biologically active molecule comprises mRNA, guide RNA, crRNA, tracrRNA, tRNA, ssDNA, dsDNA, cDNA, or a combination thereof.

12. The self-assembled nanomaterial of claim 1, wherein the Janus base nanotube is present in an amount of 0.1 wt % to 99.9 wt % based on the total weight of the self-assembled nanomaterial.

13. The self-assembled nanomaterial of claim 1, wherein a concentration of the Janus base nanotube in the self-assembled nanomaterial is 1 μg/mL to 1 g/ml.

14. The self-assembled nanomaterial of claim 1, wherein pH of the self-assembled nanomaterial is from 1 to 10.

15. The self-assembled nanomaterial of claim 1, further comprising an extracellular matrix (ECM) molecule.

16. The self-assembled nanomaterial of claim 15, wherein the ECM molecule comprises hydroxyapatite, fibronectin, Matn1, Matn3, laminin, cartilage oligomeric matrix protein, a collagen, elastin, vitronectin, fibrillin, perlecan, fibrinogen, osteonectin, tenascin, thrombospondin, an intercellular adhesion molecule (ICAM1-5), an integrin, a proteoglycan, a glycoprotein, or a combination thereof.

17. A self-assembled nanopiece (or Janus Base Nanopiece (JBNP)) comprising the self-assembled nanomaterial of claim 1 and an agent (e.g., therapeutic agent).

18. The self-assembled nanopiece of claim 17, wherein the self-assembled nanomaterial or self-assembled nanopiece comprises, or has attached thereto, a targeting molecule.

19. The self-assembled nanopiece of claim 17, wherein the agent or therapeutic agent is or includes a nucleic acid vector (e.g., a gene delivery agent (such as, DNA, plasmid, siRNA, miRNA, shRNA)), a protein, a peptide, or a small molecule.

20. An injectable pharmaceutical composition comprising the self-assembled nanomaterial of claim 1, or the self-assembled nanopiece of claim 17, and a pharmaceutically acceptable carrier.
